SEQUENCE LISTING (13 sequences)

SEQ ID NO: 1 - DNA, 3762 bp, Homo sapiens
  CDS (25)..(432)
gagaaaccgagtcactgtgaaaagatgggaaattatctcctgcgaaaactcagttgcctgggagagaatcaaaagaagcccaagaaaggaaacccagatgaggaaagaaaacggcaggaaatgactacatttgaaagaaaacttcaagatcaagataagaaaagccaagaagtttcatccacttctaatcaggaaaacgagaatggcagtggttctgaagaagtgtgctacactgtcattaatcacatcccccatcagagatcctccctgagctccaatgatgatggctatgagaacattgactccctcacaaggaaagtgagacagtttagagaaaggtcagagacagaatatgcccttcttaggacttctgttagtaggccttgttcctgcacccatgagcatgattatgaagttgtgtttccacactaaaatcctcaagctgctttatcaccttccagcaatgaagacaatgcagaatagcagactctggcgaagttgttcaccctgagcagtgcatgaaacattcctttctggctaaagtttagaaatattatcttattatatatccttaggcaactctgatatgtggcatctctgtggcttaggtgaaatcatagaaattgacacaatgacctaaaatattctatgtgtttttgcttgtaaagtttgaggacatggaggtgataaaaaaaactttcttaggacaataatgtaaaatgaaaataaatttctaatccccctgactaactgaatggaccctcttctaggccaaagagacctcagatgaacctgaaagactgaattctggccatgataggaagggaggtgagacacaccttgttataccccttcccttttggagtttatgcacaagtgaccaggatgagtcataagactgatgaaatagactgattgtggcaataagagtcccaattccaacctgactctggtgtagatcacacactgtctgagggattccatctatgagactttgtctacataacagagaccttggtttccacaacccctttattttagctaaagcattcttttctactgacttcttaagtctttagacaaagcttaactctttcaaccaattgccaatcagacaaactttgaatctacctatgacctgtaagctctctcctgcttcaagatcttgcctctttaagctgaaccgatgtgcactttccatttaatgatttatgtctttgcttgtaactcctgtctccctaaaatgtataaaagtaaacggtgacctgaccacctcaggcacactttctcaggacctcctgagagtgtatcccaggccatggtaagtcatgttggctcagaatcaacctctttaaatattttacagaatttgggttttggttaccaataagtctccacaaatatatgtccaagaatcttcaattccaagcctgctcaccaaatttcaaatgccaacatctccccatccaattacctatttcatctttgaggtgtaatctactcaataaactgtgtaagaccagtgaccagaccctttgctaacctgacatttacttcaatttttctttttctatgtactggatatttttgcatataaacttgcagtaatagttcaaaaattaatagtttttgacattggcttttctgagaagagaaattgaaagtgtcacaaaataaaaaaagatgaaatgaagcatatataattgtcaattttttcaattttctagtcaacagagaatcgaaggattctgttcaaatattagtaaaaattgaaaataaacttgtgcttatattttgtttgcaacacactagttaatttaacctgtgactagttatctctaccgaaggtggatgtgtagtttctggttttaaaattcaagcaaactggaaaataatccatctaattatgctttctttcccaagaagttttttaatgatatgccagcttcctaatttggagacaaaagccttaattgacaatgcattcattatatatttttttgtatagttacagtatacgagttgagtatcccttagatgagatgcttgggaccagaagtgttttggatttcagatttatttttggattttggaatatttccatacatataatgagagagttggaaaatgggattcaagtctaatcataaaattcacttatgtttgatatacaccttatctgaatagcctgaaggtaattttatacaatattttaaataattttatgcctgaaacagagtttgcgcacattggaccatcagaaagcagaagtgtcactatttcaagtcagtgctcaaaaagtttcagatgttaagctggtgatgcagttcatgccagtgatccgagtactttgggaagccaagacaggtggatctcttgagcccaggagtttgaggccagactgcacaacacagtgagacctcgtttctacaaataattaaaaaattagccaggtgtggtggtgcacacctgtagtcccaggtactcaggaggctgaggtagtaggattgtttgagactgggaggttgaggctgaactgagccaggatcttgccaccacattccagcttgggcaacagagtgagaccctgtctcaaaaaaaaaaaaaagtttcagattttggagcatttcagatcttcagattagggattttcaacctgtactgaccttttagtcattgacaagcattaatcaataggtggactccagataactcatttgctgtatacacattttgcctctctattcaacgaattcttatgccctcttgtggtgattttaatgtgcggaagggaaacaatagaaattttgcaattctagaaaagtcattctgtcaaaatatgtcagtcctgtagatattagccaattttaggaaaatgacaaaattttttacttttcgtctgcctttgtagctgttttatgatataaataccttatttgtaataaaattaattttaatttgagtaacaatctggaattatcagagaaggggcaagcaataggttaataaacagtattgattggtagaaggaacgttgaaatccaagagcatcaatgtcttctggtggttcaccataagccacagcagatgtcttaatctttccgagatctagtttttcagcaaagcaggatttaagaaatgtaactatcttatgtggttatgaagaacaatagaatcattgctgtataagtgctttttaacctgtaaattttgtgaagcttatcttttatgcatataaatatttgaacattttacattgtttatatttttaatcagttttactcaagtgtgattatatacaagaaaatgtaaccactgtaagggtagagttataagaattttgtcaaatgtattcacccatgtagtcacctccttatgaagagacagaacacgtacatcctcccagaaagttccacagtgctccttttccctgagtttcaccagtcctggcaaccaatgatctgcttcgtataattataactgttctagatatttgtagcaatgtaccctttccatatttattttgtgtgtgtaaggcttcttttagtcattataatatttttgagattcatctatgtttaatgttctatcagtagttgtacatcttacttgtctcagcatatcaccatatagatatactataatttgttaatctaatcactgatggatatgtaggatatttaagtttttgacattatgaataaagtggctataaatgaaaaaaaaaaaaaaaaaaaaaaaaaaaaaaaaaaaaaaaaaaaa

SEQ ID NO: 2 - protein, 135 aa, Homo sapiens
MGNYLLRKLSCLGENQKKPKKGNPDEERKRQEMTTFERKLQDQDKKSQEVSSTSNQENENGSGSEEVCYTVINHIPHQRSSLSSNDDGYENIDSLTRKVRQFRERSETEYALLRTSVSRPCSCTHEHDYEVVFPH

SEQ ID NO: 3 - DNA, 3663 bp, Homo sapiens
  CDS (294)..(746): First ORF
cccacgcgtccggaaccagcctgcacgcgctggctccgggtgacagccgcgcgcctcggccaggatctgagtgatgagacgtgtccccactgaggtgccccacagcagcaggtgttgagcatgggctgagaagctggaccggcaccaaagggctggcagaaatgggcgcctggctgattcctaggcagttggcggcagcaaggaggagaggccgcagcttctggagcagagccgagacgaagcagttctggagtgcctgaacggccccctgagccctacccgcctggcccactatggtccagaggctgtgggtgagccgcctgctgcggcaccggaaagcccagctcttgctggtcaacctgctaacctttggcctggaggtgtgtttggccgcaggcatcacctatgtgccgcctctgctgctggaagtgggggtagaggagaagttcatgaccatggtgctgggtgagtcactacatcctccttccttcctgttccagatacatgccacctggcatgtgggacaggagtacctctgccctgggagctgcttggagggagaggtggtctgctgggaaggcattgctgggcaggagggtgaccctgggctgagggggcacaccaagagaaagaagagaataccaaggacataccccagtcacctctggatccctggtcctgcacagagcctggctcataggagacactggagaaatgctcctaacctttggctagcccttttataatttatagcgattatctcatttaatgcttacaaccaccatttgaggtgatccattttacagagaaggaagcagaggcttttaagaggttaggtaagtcttagccaaagccaaatagcagctgaacagtagagctgggactccatcaaggtctcccagccggagcttgctcctacccctaggacaaggggtggactcctgactctgcagataaattctacaaaagccacagaaggcaagtagtaaccattgtgtgacaacccctcacccccaggaagaggggcccctgtgaggattgcaggctctggagtcacactgcttgttgaaacgctgcctcttaccctccctaggtctgcgcctttgaataagtatcacttcttagttgctccatgcctcagtttgtccatctgaaaatgggggcatctgtaatgcctgtgttatgaggagtaaattacagcatccctgtgaagacgtagcacagtgtcgagtacggaatgttatttccatccttctcacggagcttggttccccttccccttgccctttacttgtcccagccattgactcatactacttcccttcttgcaggcattggtccagtgctgggcctggtctgtgtcccgctcctaggctcagccagtgaccactggcgtggacgctatggccgccgccggcccttcatctgggcactgtccttgggcatcctgctgagcctctttctcatcccaagggccggctggctagcagggctgctgtgcccggatcccaggcccctggagctggcactgctcatcctgggcgtggggctgctggacttctgtggccaggtgtgcttcactccactggaggccctgctctctgacctcttccgggacccggaccactgtcgccaggcctactctgtctatgccttcatgatcagtcttgggggctgcctgggctacctcctgcctgccattgactgggacaccagtgccctggccccctacctgggcacccaggaggagtgcctctttggcctgctcaccctcatcttcctcacctgcgtagcagccacactgctggtggctgaggaggcagcgctgggccccaccgagccagcagaagggctgtcggccccctccttgtcgccccactgctgtccatgccaggccctaagcctggagctcccttccctaatggacacgtgggtgctggaggcagtggcctgctcccacctccacccgcgctctgcggggcctctgcctgtgatgtctccgtacgtgtggtggtgggtgagcccaccgaggccagggtggttccgggccggggcatctgcctggacctcgccatcctggatagtgccttcctgctgtcccaggtggccccatccctgtttatgggctccattgtccagctcagccagtctgtcactgcctatatggtgtctgccgcaggcctgggtctggtcgccatttactttgctacacaggtagtatttgacaagagcgacttggccaaatactcagcgtagaaaacttccagcacattggggtggagggcctgcctcactgggtcccagctccctgctcctgttagccccatggggctgccgggctggccgccagtttctgttgctgccaaagtaatgtggctctctgctgccaccctgtgctgctgaggtgcgtagctgcacagctgggggctggggcgtccctctcctctctccccagtctctagggctgcctgactggaggccttccaagggggtttcagtctggacttatacagggaggccagaagggctccatgcactggaatgcggggactctgcaggtggattacccaggctcagggttaacagctagcctcctagttgagacacacctagagaagggtttttgggagctgaataaactcagtcacctggtttcccatctctaagccccttaacctgcagcttcgtttaatgtagctcttgcatgggagtttctaggatgaaacactccaccatgggatttgaacatatgaaagttatttgtaggggaagagtcctgaggggcaacacacaagaaccaggtcccctcagcccacagcactgtctttttgctgatccacccccctcttaccttttatcaggatgtggcctgttggtccttctgttgccatcacagagacacaggcatttaaatatttaacttatttatttaacaaagtagaagggaatccattgctagcttttctgtgttggtgtctaatatttgggtagggtgggggatccccaacaatcaggtcccctgagatagctggtcattgggctgatcattgccagaatcttcttctcctggggtctggccccccaaaatgcctaacccaggaccttggaaattctactcatcccaaatgataattccaaatgctgttacccaaggttagggtgttgaaggaaggtagagggtggggcttcaggtctcaacggcttccctaaccacccctcttctcttggcccagcctggttccccccacttccactcccctctactctctctaggactgggctgatgaaggcactgcccaaaatttcccctacccccaactttcccctacccccaactttccccaccagctccacaaccctgtttggagctactgcaggaccagaagcacaaagtgcggtttcccaagcctttgtccatctcagcccccagagtatatctgtgcttggggaatctcacacagaaactcaggagcaccccctgcctgagctaagggaggtcttatctctcagggggggtttaagtgccgtttgcaataatgtcgtcttatttatttagcggggtgaatattttatactgtaagtgagcaatcagagtataatgtttatggtgacaaaattaaaggctttcttatatgtttaaaaaaaaaaaaaaaaaaaaaaaaa

SEQ ID NO: 4 - protein, 150 aa, Homo sapiens
MVQRLWVSRLLRHRKAQLLLVNLLTFGLEVCLAAGITYVPPLLLEVGVEEKFMTMVLGESLHPPSFLFQIHATWHVGQEYLCPGSCLEGEVVCWEGIAGQEGDPGLRGHTKRKKRIPRTYPSHLWIPGPAQSLAHRRHWRNAPNLWLALL

SEQ ID NO: 5 - protein, 326 aa, Homo sapiens
MLFPSFSRSLVPLPLALYLSQPLTHTTSLLAGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCQALSLELPSLMDTWVLEAVACSHLHPRSAGPLPVMSPYVWWWVSPPRPGWFRAGASAWTSPSWIVPSCCPRWPHPCLWAPLSSSASLSLPIWCLPQAWVWSPFTLLHR

SEQ ID NO: 6 - DNA, 3743 bp, Homo sapiens
  CDS (377)..(1951)
ggcgggcagggaggcggcgggcgccgaggagacgcagcggcggcggtggtggcggtggtggcggcggtggcggcggcggcgcctggtctcggccgcagcctctgcgtcccgcccgctctgggtcgcccctgccagccccgcgcgcggctcgcctcccgccgcggcccgcagcctgcggcgcccgagctccgagggcgcttccccgggcgctgggcccgaggcggccaagacggcgggccggccgggcacggggcggcgggcgctgtgctgcgggctgcgctggtggggggcggcgggccgcggcgcgggcccagggcccggcgtgtgcggctgggaggcactgtggggggcgggcgcgcgggcggcggccggggccatggccgagggcggcgggggcgcgcggaggagggcgccggcgctgctcgaggctgcccgcgcgcgctacgagagcctgcacatctcggacgacgtgttcggcgagtccggcccggacagcggcgggaaccccttctacagcacctcggccgcctcgcgctcctcctcggccgcctcctcggacgacgagcgcgagcccccgggacccccaggggccgccccgccaccgccccgcgccccggacgcacaggagccggaggaggacgaggccggcgcgggctggagcgcagcgctgcgggaccgcccgcccccgcgcttcgaggataccggcggtcccacccgaaagatgccccccagcgccagtgccgtggacttcttccagctctttgtcccagacaacgtcctcaagaacatggtggtgcagacaaacatgtatgccaagaagttccaggagcggtttgggagcgacggagcctgggtggaggtgacgctgacggagatgaaggcgttcctgggctacatgatctccaccagcatctcccactgcgagtccgtcctcagcatctggagcggaggcttctacagcaaccgcagcctcgccctcgtcatgagccaggcccgcttcgagaagatcctcaagtacttccacgtcgtggccttccgctccagccagaccacgcacgggctctacaaggtccagcccttcctcgactccctgcagaacagcttcgactctgccttcaggccttcccaaacccaggtgctacatgaacccctgatcgatgaggatcctgtattcattgccacgtgcacagagcgggagctgcgaaagaggaaaaagcggaaattcagcctctgggtcagacaatgttcttccactggcttcatcatccagatttatgtccacctgaaggaaggtgggggcccagatggcctggatgcgctgaagaataagccccagctccacagcatggtggccaggagcctgtgccggaacgcggcaggcaagaactacatcattttcacggggcccagcatcaccagcctgacgctgtttgaagagtttgagaagcaagggatttactgctgcggcttgctccgcgcgcggaagagtgactgcaccggcctcccactgtccatgctgaccaacccagccacacccccggcccggggccagtaccaaatcaagatgaaggggaacatgtccttgatctgctggtacaacaaaggacacttccgcttcctgaccaacgcctactccccggtgcagcagggagtcatcatcaaaaggaagagtggggagatcccatgccccttggccgtggaggcgtttgccgctcacctgagctacatctgcagatacgatgacaaatacagcaagtatttcatttctcataaaccaaacaagacctggcagcaggtgttctggttcgccatcagcatcgccatcaacaatgcctacatcctgtacaaaatgtcagacgcctaccacgtgaagaggtacagccgggcgcagtttggagagagactcgtcagagagctgctgggcttggaggatgcctctccgacccactgatgctgggggcgcaggactcggtcaagggaggggcaagaggaggaggagagcctgccgttccaacttgcccatcagagacccggacacggcctggtgtgtggcttgctgcctgggagggatgcacagggcctctggagggacaggatggacctggtcagaggacggttgctgtcctcatttgcattccaagaagagcatgtcctccctcgagaaacagtgccgccggtgtgatgagcacttacacccacgttctcaagggcagattctctcatgacatccgtggagcttgcgaggcagcgtggactggtgactgtgaaggaaggcccccgtggtagaatgagctggagcacgctctaagagagatgcctgcttcctaaagatctacagcaatctgggacgtggttcaagttcaagacttgaaggaagcaaagacgccctgcatggttacaatggctcaggtgtcaggggaggccggagggttttccagcatttgcctcatgccagcacctttgaaccggtctcttagaagaagacacacatcctgggtgtacagtggtgaaatggggagtgggtgcccattctgaaaaacgaggcattcctgctcattccctctgcttagctggtgggcaggggagagagggaaatgccaaaaacttggagtgaaggatgatgctattttttatttttaaatatatcttcaggttattttcttactgttgcttcagatctaatgtaaaaggcagatgtcccctcctctccacccccgacgctgaccccggcctcagtcacggctctttgcatgatcacagttctgtgttctggcctgtggcagggccgggaagggccgctggcttccgaacagacgtggttgctctccacgaggcgcatggggagcccgcgggccctaagctttgtcgcagatgtcatcattggcagaattacttgtcttgaaaaataagtagcattgctgaaacacacaaccgaattctctacgatggccatttgctcattgtctttcctctgtgtgtagtgagtgaccctggcagtgtttgcctgctcagagtggcccctcagaacaacagggctggccttggaaaaaccccaaaacaggactgtggtgacaactctggtcaggtgtgatttgacatgagggccggaggcggttgctgacggcaggactggagaggctgcgtgcccggcactggcagcgaggctcgtgtgtcccccaggcagatctgggcactttcccaacccaggtttatgcgtctccagggaagcctcggtgccagagtggtgggcagatctgaccatccccacagaccagaaacaaggaatttctgggattacccagtcccccttcaacccagttgatgtaaccacctcattttttacaaatacagaatctattctactcaggctatgggcctcgtcctcactcagttattgcgagtgttgctgtccgcatgctccgggccccacgtggctcctgtgctctagatcatggtgactcccccgccctgtggttggaatcgatgccacggattgcaggccaaatttcagatcgtgtttccaaacacccttgctgtgccctttaatgggattgaaagcacttttaccacatggagaaatatatttttaatttgtgatgcttttctacaaggtccactatttctgagtttaatgtgtttccaacacttaaggagactctaatgaaagctgatgaattttcttttctgtccaaacaagtaaaataaaaataaaagtctatttagatgttaaaaaaaaaaaaaaa

SEQ ID NO: 7 - protein, 524 aa, Homo sapiens
MAEGGGGARRRAPALLEAARARYESLHISDDVFGESGPDSGGNPFYSTSAASRSSSAASSDDEREPPGPPGAAPPPPRAPDAQEPEEDEAGAGWSAALRDRPPPRFEDTGGPTRKMPPSASAVDFFQLFVPDNVLKNMVVQTNMYAKKFQERFGSDGAWVEVTLTEMKAFLGYMISTSISHCESVLSIWSGGFYSNRSLALVMSQARFEKILKYFHVVAFRSSQTTHGLYKVQPFLDSLQNSFDSAFRPSQTQVLHEPLIDEDPVFIATCTERELRKRKKRKFSLWVRQCSSTGFIIQIYVHLKEGGGPDGLDALKNKPQLHSMVARSLCRNAAGKNYIIFTGPSITSLTLFEEFEKQGIYCCGLLRARKSDCTGLPLSMLTNPATPPARGQYQIKMKGNMSLICWYNKGHFRFLTNAYSPVQQGVIIKRKSGEIPCPLAVEAFAAHLSYICRYDDKYSKYFISHKPNKTWQQVFWFAISIAINNAYILYKMSDAYHVKRYSRAQFGERLVRELLGLEDASPTH

SEQ ID NO: 8 - DNA, 1998 bp, Homo sapiens
  CDS (162)..(635): 1st open reading frame
tgcatcaggactttaccagataatgttcttccagatctgaaaaggaaaatgcctaaaagagcttccaaactcatttttgaataatactaggctacaaagaattacactgtgaattcattaagggtaacaccaaatcactaaacagcactgtttgtacagaaatgtcgaaaagctgtggaaataatttagcggccatttctgtaggaatttcgcttcttttactcttagtggtttgtggaattgggtgtgtttggcactggaaacaccgtgttgccacacgatttaccttaccgaggtttttacaaaggagaagcagcaggagaaaagtctgtactaaaacattcttgggcccccgcatcattggcttaaggcatgaaatctcagttgaaacccaagaccacaaatctgctgtcaggggaaataacacacacgacaactatgaaaatgtggaagcaggtcctcccaaagctaaaggaaaaaccgataaggaactatatgaaaacacagggcagtctaatttcgaggagcatatctatggaaatgagacatcttctgactattataacttccagaagcctcgtccttctgaagttcctcaagatgaagatatatacattcttccagattcatattagcttttcaaaatattgacttttgttattggatgataaatattcactgtaatttttcaacagcaaagacaaggaatcaaactaaatgttgatcaactgtagactggataaagaaaatgtggtacacatacaccatagaatattatgcagccgtaaaaaaagaacaaaactaacatgggaacagaaaatcaaataccacatattctcacttaaaagtgggagctaaataataagaacacatggagagaaggagaggaacaacagacactggggcctacttgagggaggacagtggaaggagggagaggttcagggaaaaaaaaaatatcaggtactatgcttagtacacacatgatgaaataatctgtacaccaaacccccaagtcacaagtgttcctacataacaaacctgaacatgtacccctgaacataaaattataattaaaatattaaaaataattcactgtgatttttattgtactgatgccattcttaatcaagttctgataagtggatggtctctgcctatctccacctttctgaatcctatgtgtatcgctgtggattaattctagatatcttctccaccctccttgcaccagactaaatctgtattatgtgatattgattcttccttctaaatattacccgttatctctttcctttatttctaccattatctttatctggctcagaattattgtcataggctcctaactgttcctcctgcttctagtttctacccactcaatcaattaccgatggtgttgccagatttatcttcagaaaatattcctaacagccacattatttctttcacttaaaatgttttaatgccccctctttgcaaaagacataatacccataatttgaactccaaaatttatggttttccacaattggttccaattcacttttccagtgacttctcttactatctctcatttctttgccttcagcagaatcatcttaaaacctgccaaacttatccttccttcacagctttgcttttctgcctcttctctcaagcctgcttcagatcataagttcttccacacatctcctgaatcactccaaacccgcatttacctttttattttctgatataagctttgatgcctcttcaattcttaggacatttaaacatatgaatgttgccacagcattttattacctagcttcatatgaaaatgtcttaaattcccacctaaatgaaaagaaactgcccaaatgcctagaacatcacataaggcactaaatgcctcatgttttactgacgggaattgaattgtacattttgctgagtagttttgagaaaaaaatctaataaattcatctgttattcatccataaaaaaaaaaaaaaa

SEQ ID NO: 9 - protein, 157 aa, Homo sapiens
MSKSCGNNLAAISVGISLLLLLVVCGIGCVWHWKHRVATRFTLPRFLQRRSSRRKVCTKTFLGPRIIGLRHEISVETQDHKSAVRGNNTHDNYENVEAGPPKAKGKTDKELYENTGQSNFEEHIYGNETSSDYYNFQKPRPSEVPQDEDIYILPDSY

SEQ ID NO: 10 - DNA, 1998 bp, Homo sapiens; identical to SEQ ID NO: 8
  CDS (373)..(651): 2nd open reading frame

SEQ ID NO: 11 - protein, 92 aa, Homo sapiens
MKSQLKPKTTNLLSGEITHTTTMKMWKQVLPKLKEKPIRNYMKTQGSLISRSISMEMRHLLTIITSRSLVLLKFLKMKIYTFFQIHISFSKY

SEQ ID NO: 12 - DNA, 1662 bp, Homo sapiens
  CDS (1)..(1662)
atggtccagaggctgtgggtgagccgcctgctgcggcaccggaaagcccagctcttgctggtcaacctgctaacctttggcctggaggtgtgtttggccgcaggcatcacctatgtgccgcctctgctgctggaagtgggggtagaggagaagttcatgaccatggtgctgggcattggtccagtgctgggcctggtctgtgtcccgctcctaggctcagccagtgaccactggcgtggacgctatggccgccgccggcccttcatctgggcactgtccttgggcatcctgctgagcctctttctcatcccaagggccggctggctagcagggctgctgtgcccggatcccaggcccctggagctggcactgctcatcctgggcgtggggctgctggacttctgtggccaggtgtgcttcactccactggaggccctgctctctgacctcttccgggacccggaccactgtcgccaggcctactctgtctatgccttcatgatcagtcttgggggctgcctgggctacctcctgcctgccattgactgggacaccagtgccctggccccctacctgggcacccaggaggagtgcctctttggcctgctcaccctcatcttcctcacctgcgtagcagccacactgctggtggctgaggaggcagcgctgggccccaccgagccagcagaagggctgtcggccccctccttgtcgccccactgctgtccatgccgggcccgcttggctttccggaacctgggcgccctgcttccccggctgcaccagctgtgctgccgcatgccccgcaccctgcgccggctcttcgtggctgagctgtgcagctggatggcactcatgaccttcacgctgttttacacggatttcgtgggcgaggggctgtaccagggcgtgcccagagctgagccgggcaccgaggcccggagacactatgatgaaggcgttcggatgggcagcctggggctgttcctgcagtgcgccatctccctggtcttctctctggtcatggaccggctggtgcagcgattcggcactcgagcagtctatttggccagtgtggcagctttccctgtggctgccggtgccacatgcctgtcccacagtgtggccgtggtgacagcttcagccgccctcaccgggttcaccttctcagccctgcagatcctgccctacacactggcctccctctaccaccgggagaagcaggtgttcctgcccaaataccgaggggacactggaggtgctagcagtgaggacagcctgatgaccagcttcctgccaggccctaagcctggagctcccttccctaatggacacgtgggtgctggaggcagtggcctgctcccacctccacccgcgctctgcggggcctctgcctgtgatgtctccgtacgtgtggtggtgggtgagcccaccgaggccagggtggttccgggccggggcatctgcctggacctcgccatcctggatagtgccttcctgctgtcccaggtggccccatccctgtttatgggctccattgtccagctcagccagtctgtcactgcctatatggtgtctgccgcaggcctgggtctggtcgccatttactttgctacacaggtagtatttgacaagagcgacttggccaaatactcagcgtag

SEQ ID NO: 13 - protein, 553 aa, Homo sapiens
MVQRLWVSRLLRHRKAQLLLVNLLTFGLEVCLAAGITYVPPLLLEVGVEEKFMTMVLGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCRARLAFRNLGALLPRLHQLCCRMPRTLRRLFVAELCSWMALMTFTLFYTDFVGEGLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSA

(19) United States
(12) Patent Application Publication
(10) Pub. No.: US 2003/0166881 A1
(43) Pub. Date: Sep. 4, 2003

(54) Novel genes associated with allergic hypersensitivity and mast cell activation

(76) Inventors: Karl Nocka, Harvard, MA (US); Gregorio Pirozzi, Gaithersburg, MD (US); Richard Einstein, Gaithersburg, MD (US)

Correspondence Address: MORGAN LEWIS & BOCKIUS LLP, 1111 PENNSYLVANIA AVENUE NW, WASHINGTON, DC, 20004, US

(21) Appl. No.: 10/005,907

(22) Filed: Dec. 7, 2001

Related U.S. Application Data
(60) Provisional application No. 60/251,835, filed on Dec. 8, 2000.
(60) Provisional application No. 60/275,479, filed on Mar. 14, 2001.
(60) Provisional application No. 60/279,115, filed on Mar. 28, 2001.
(60) Provisional application No. 60/280,143, filed on Apr. 2, 2001.

(51) Int. Cl. C07H 21/02; C07H 21/04

(52) U.S. Cl. 536/23.1

(57) ABSTRACT

The invention relates generally to the changes in gene expression in mast cells during maturation and in tissues removed from patients with urticaria or allergic hypersensitivity relative to gene expression in mast cells removed from normal subjects. The invention specifically relates to four novel gene families which are differentially expressed in mast cells and allergic hypersensitivity diseases, such as urticaria, compared to normal tissues.

### CROSS-REFERENCE TO RELATED APPLICATIONS

This application claims priority to U.S. provisional applications 60/251,835, filed Dec. 8, 2000; No. 60/275,479, filed Mar. 14, 2001; No. 60/279,115, filed Mar. 28, 2001 and 60/280,143, filed Apr. 2, 2001, all of which are incorporated herein in their entirety by reference.

### FIELD OF THE INVENTION

The invention relates generally to the changes in gene expression in mast cells and tissues removed from patients with allergic hypersensitivity. The invention specifically relates to four novel gene families that are differentially expressed in mast cells compared to other tissues and in resting mast cells versus activated mast cells.

### BACKGROUND OF THE INVENTION

Allergic Hypersensitivity

The inflammatory response characteristic of allergic or hypersensitivity reactions can be elicited by extrinsic antigens such as pollen, dust, food, and chemicals in the environment. There are four main classes of hypersensitivity reactions, which are distinguished by the type of immune cells and antibodies involved and the pathologies produced. In the most common IgE-dependent allergic reactions, the inflammatory response involves mast cell degranulation, or release of the granules, triggered by allergen interaction with IgE molecules on the mast cell surface. Present in large numbers in epithelial tissue, mast cells have high-affinity IgE receptors on their surface.

Inhaled allergens initiate respiratory allergies such as allergic rhinitis, hay fever and asthma, while ingested allergens may cause food allergies. Certain allergens (antibiotics, insect venoms) may cause life-threatening anaphylactic reactions.

The granules of the mature mast cell contain mediators of allergies, such as histamine, heparin, and chemotactic factors for inflammatory cells and proteases. Newly formed lipid mediators, such as leukotrienes and prostaglandins are also rapidly synthesized and released from mast cells upon activation. Preformed and de novo synthesized mediators released on the mucosal surfaces cause the symptoms associated with allergic rhinitis or hay fever, for example itchy watery eyes, runny nose, hives and sneezing. Many of the symptoms associated with asthma are also associated with the direct effects of mediators released from the mast cell as well as indirectly through other cells that are recruited to the lung by these mediators. These mediators also contract the respiratory smooth muscles, limit breathing, and may cause an anaphylactic reaction, which can be fatal. Release of these mediators in the skin causes edema, erythema, and wheal formation, i.e., urticaria.

Urticaria is a skin condition characterized by the appearance of intensely itching wheals or welts with elevated centers and a surrounding area of erythema (redness). Wheals are usually distributed over the trunk and extremities of the body, but they may occur on any epithelial or mucosal surface. A related skin condition, angiodema, with similar, but non-pruritic, sores, affects deeper levels of skin tissues. It is estimated that 10-20% of the population, usually children, suffers from urticaria or angiodema or both simultaneously at one time or another (Frank, M. M., Cecil Textbook of Medicine, 20^{th} Ed., Bennet and Plum eds., chap. 19, pp. 1408-1412, W. B. Saunders Co., Philadelphia, 1996). In about 70% of urticaria/angiodema cases, the cause of the disease is not found. Typical sources, however, are ingested allergens from foods or drugs, such as non-steroidal anti-inflammatories, or autoimmune-type allergens, such as antithyroid, anti-IgG or anti-IgE autoantibodies. Other causes include physical factors, e.g., heat, cold, pressure and sun.

Treatment of Allergies and Asthma

Conventional therapeutic compounds, such as antihistamines, prevent histamine from binding to H1 receptors. Antihistamines may be taken to block the effect of histamine released from mast cell granules, but they have no effect on the activities of the other co-released vasoactive compounds. Cromolyn sodium and nedocromil are effective in some patients and may block mast cell degranulation and, therefore, the release of histamine as well as other mediators from mast cells. For urticaria and angiodema, the most common and most effective treatment to date is administration of antihistamines or glucocorticoids, although, where antihistamines prove ineffective, the symptoms can persist for years. Lipoxygenase inhibitors or leukotriene antagonists may specifically block the effects of leukotrienes released from mast cells. Other agents such as glucocorticoids, theophylline, and beta-agonists play important roles in the control of asthma. They have all been shown to have an inhibitory effect on either mast cell development or activation. A new approach of targeting IgE directly with monoclonal-antibody therapy has been found to be effective in some patients with rhinitis as well as asthma and has demonstrated the importance of this trigger on the mast cell in these diseases.

Cellular and Molecular Events Involved in Mast Cell Function and Activation

Much is known about the biochemical pathways and proteins that are involved in the cascade of events that follow activation of rodent mast cells through the high affinity IgE receptor. Most of this information has been determined through studies of the rat mast cell line RBL-2H3, freshly isolated rat peritoneal mast cells, or murine cultured bone marrow derived mast cells. The properties of these rodent mast cells are unique and differ significantly from the human tissue mast cells in terms of stage of maturity and response to positive and negative regulators. The culture of human cord blood mast cells has been described; however, these mast cells are not ideal for studying signal transduction due to their immature phenotype.

While the changes in the expression levels of a number of individual genes have been identified, i.e., cytokines and chemokines, the investigation of the global changes in gene expression has only recently been reported in cord blood human mast cells. Accordingly, there exists a need for the investigation of the changes in global gene expression levels in mast cells from normal and atopic donors, as well as the need for the identification of new molecular markers associated with mast cell maturation, activation, degranulation and regranulation. Furthermore, if intervention is expected to be successful in preventing or reducing allergic hypersensitivity, means of accurately assessing mast cell maturation and the early stages of activation need to be established. One way to accurately assess the early cellular events involved in mast cell activation is to identify markers that are uniquely associated with the process. Likewise, the development of therapeutics to prevent or stop IgE-mediated allergic reactions relies on the identification of genes responsible for mast cell maturation, activation and de- and regranulation.

### SUMMARY OF THE INVENTION

The present invention is based on the discovery of four new gene families that are differentially expressed in mast cells that have been activated through the IgE receptor. A second gene-selection criterion is the high level of expression of this gene family in mast cells as compared to non-mast cell populations or whole tissues.

The genes and proteins of the invention were detected in primary human mast cells. These cells were derived from precursors initially isolated from adult peripheral blood which mature into mast cells over 4-6 weeks in culture under defined culture conditions. The cells respond to IgE and antigen in a similar manner as mature tissue mast cells by degranulation and histamine release. Samples were collected from a patient with urticaria and from umbilical cord blood. Mast cells were purified from these populations and compared to mast cells isolated from normal donors under a variety of conditions. The cells isolated from the urticaria patient are thought to be highly sensitive to stimuli, i.e. have an activated morphology in the absence of a stimulus, during early stages of maturation. As described below, the differential expression of gene species has been studied using these cells.

The present invention is based on the discovery of new gene families that are differentially expressed in allergic hypersensitivity, for example in activated compared to normal, unactivated cells. The invention includes isolated nucleic acid molecules selected from the group consisting of an isolated nucleic acid molecule that encodes the amino acid sequence of SEQ ID NO: 2, 4, 5, 7, 9, 11 or 13, an isolated nucleic acid molecule that encodes a fragment of at least 6 amino acids of SEQ ID NO: 2, 4, 5, 7, 9, 11 or 13, an isolated nucleic acid molecule which hybridizes to the complement of a nucleic acid molecule comprising SEQ ID NO: 1, 3, 6, 8, 10 or 12 and an isolated nucleic acid molecule which hybridizes to the complement of a nucleic acid molecule that encodes the amino acid sequence of SEQ ID NO: 2, 4, 5, 7, 9, 11 or 13. Nucleic acid molecules of the invention may encode a protein having at least about 35%, 40%, 50%, 60%, or 65% amino acid sequence identity to SEQ ID NO: 2, 4, 5, 7, 9, 11 or 13, preferably at least about 70% or 75% sequence identity, more preferably at least about 80-85% sequence identity, and even more preferably at least about 90% or 95% sequence identity to SEQ ID NO: 2, 4, 5, 7, 9, 11 or 13.

The present invention further includes the nucleic acid molecules operably linked to one or more expression control elements, including vectors comprising the isolated nucleic acid molecules. The invention further includes host cells transformed to contain the nucleic acid molecules of the invention and methods for producing a protein comprising culturing a host cell transformed with a nucleic acid molecule of the invention under conditions in which the protein is expressed.

The invention further provides an isolated polypeptide selected from the group consisting of an isolated polypeptide comprising the amino acid sequence of SEQ ID NO: 2, 4, 5, 7, 9, 11 or 13, an isolated polypeptide comprising a functional or antigenic fragment of at least 6 amino acids of SEQ ID NO: 2, 4, 5, 7, 9, 11 or 13, an isolated polypeptide comprising conservative amino acid substitutions of SEQ ID NO: 2, 4, 5, 7, 9, 11 or 13 and an isolated polypeptide comprising naturally occurring amino acid sequence variants of SEQ ID NO: 2, 4, 5, 7, 9, 11 or 13. Polypeptides of the invention also include polypeptides with an amino acid sequence having at least about 35%, 40%, 50%, 60%, 65%, 70% or 75% amino acid sequence identity with the sequence set forth in SEQ ID NO: 2, 4, 5, 7, 9, 11 or 13, more preferably at least about 80%, even more preferably at least about 90%, and most preferably at least about 95% sequence identity with the sequence set forth in SEQ ID NO: 2, 4, 5, 7, 9, 11 or 13.

The invention further provides an isolated antibody or antigen-binding fragment that specifically binds to a polypeptide of the invention, including monoclonal and polyclonal antibodies.

The invention further provides methods of identifying an agent which modulates the expression of a nucleic acid encoding a protein of the invention, comprising: exposing cells which express the nucleic acid to the agent; and determining whether the agent modulates expression of said nucleic acid, thereby identifying an agent which modulates the expression of a nucleic acid encoding the protein.

The invention further provides methods of identifying an agent which modulates at least one activity of a protein of the invention, comprising: exposing cells which express the protein to the agent; and determining whether the agent modulates at least one activity of said protein, thereby identifying an agent which modulates at least one activity of the protein.

The invention further provides methods of identifying binding partners for a protein of the invention, comprising: exposing said protein to a potential binding partner; and determining if the potential binding partner binds to said protein, thereby identifying binding partners for the protein.

The present invention further provides methods of modulating the expression of a nucleic acid encoding a protein of the invention, comprising administering an effective amount of an agent which modulates the expression of a nucleic acid encoding the protein. The invention also provides methods of modulating at least one activity of a protein of the invention, comprising administering an effective amount of an agent which modulates at least one activity of the protein.

The present invention further includes non-human transgenic animals modified to contain the nucleic acid molecules of the invention or mutated nucleic acid molecules such that expression of the encoded polypeptides of the invention is prevented.

The invention further provides methods of diagnosing states of IgE-mediated hypersensitivity, urticaria or mastocytosis comprising determining the level of expression of a nucleic acid molecule of the invention or polypeptide of the invention.

The invention further provides methods for the treatment or prevention of various diseases or conditions associated with mast cell activity or lack thereof. The invention provides for the use of the nucleic acid molecules or proteins of the invention, or biologically active fragments thereof, for the modulation of mast cell maturation, differentiation, growth, activation and/or mediator synthesis or release.

### BRIEF DESCRIPTION OF THE DRAWINGS

FIG. 1 shows a Northern blot, in which the expression level of SEQ ID NO: 1 (MC1, clone no. AA447527) was measured in several cell lines or cultures. Lane 1) 3-week (immature) mast cell culture; 2) activated 6-week (mature) mast cell culture; 3) resting mast cell leukemia cell line (HMC-1); 4) basophilic leukemia cell line (KU812); 5) megakaryocytic leukemia cell line (MO7e). Ribosomal 28s and 18s RNA size markers are present on the left side of the blot.

FIG. 2 shows an electronic Northern assay in which the intensity of expression of MC1 (AA447527) was measured across a panel of cultured mast cells using the Affymetrix GeneChip 42K set. The cultured mast cell age, and treatment (resting, activated or reactivated) are indicated. Urticaria=cells obtained from peripheral blood of a patient with urticaria. Act=Activation of mature (6 week old) cultured mast cells at time zero by IgE with antigen and the time periods shown indicate the time elapsed between activation and cell sampling. React.=cells that were initially activated at six weeks, allowed to recover, activated one week later, allowed to recover, and finally activated at a total of 8 weeks culture time and sampled 3 h after final activation. CordBlood=obtained from the umbilical cord at the time of birth. KL control and KL starved cells that were cultured in either control medium containing kit ligand, or in medium that did not contain the cytokine, respectively.

FIGS. 3A and 3B show an electronic Northern assay consisting of normal human tissues and a sample set consisting of all 15 samples present in FIG. 2 (15 chip sample) in which the intensity of expression of AA447527 (MC1; SEQ ID NO: 1) was measured across a panel of tissues and cell cultures using the Affymetrix human GeneChip 42K chip set. The mean intensity of expression is shown as a horizontal bar that starts at the mean value minus (−) standard deviation and stops at the mean plus (+) standard deviation. The number of data points included in each mean value is given in parentheses to the right of the tissue name.

FIG. 4 shows a hydrophobicity analysis of the polypeptide of SEQ ID NO: 2 using the methods of Goldman et al. and of Kyle-Doolittle.

FIG. 5 shows an expression assay in which the intensity of expression of N95796 (MC14; SEQ ID NO: 3) was measured across a panel of cultured mast cells using the Affymetrix GeneChip 42K set. The cultured mast cell age and treatment (resting, activated or reactivated) are indicated. KL starved=mast cells cultured in control medium containing kit ligand for 6 weeks, followed by washing and 7 hours incubation in medium without kit ligand (cytokine). Act.=Activation of mature (6 week old) cultured mast cells at time zero by IgE with antigen, and the time periods shown indicate the time elapsed between activation and cell sampling. React=cells were initially activated at six weeks, allowed to recover, activated one week later, allowed to recover, and finally activated at a total of 8 weeks culture time and sampled 3 h after final activation. Urticaria=cells cultured from peripheral blood of a patient with urticaria and allergic hypersensitivity. Cord Blood=mast cells cultured for 6 weeks from umbilical cord blood at the time of birth. Control (6 week old, mature cells)=mast cells cultured in control medium containing kit ligand for 6 weeks.

FIG. 6 shows an electronic Northern assay of normal human tissues in which the intensity of expression of N95796 (MC14) was measured across a panel of tissues and cell cultures using the Affymetrix human GeneChip 42K chip set. The mean intensity of expression is shown as a horizontal bar that starts at the mean value−standard deviation and stops at the mean=standard deviation.

FIG. 7 shows the relative expression of N95796 (MC14) using quantitative RT-PCR on RNA samples from normal mast cells, mast cells from patients with urticaria or asthma, mature cultured mast cells (unactivated, activated or reactivated, described in FIG. 5 and Example 4 below), various cell lines (KU 812, Jurkat or Mo7E), various types of white blood cells (peripheral blood lymphocytes (PBL), monocytes or eosinophils) and cord blood derived mast cells.

FIG. 8 shows a hydrophobicity analysis of the polypeptide of SEQ ID NO: 4 using the methods of Goldman et al. and of Kyle-Doolittle.

FIG. 9 shows a hydrophobicity analysis of the polypeptide of SEQ ID NO: 5 using the methods of Goldman et al. and of Kyle-Doolittle.

FIG. 10 shows a Northern blot, in which the expression level of a 3.6 kB mRNA species corresponding to SEQ ID NO: 3 (clone no. N95796) was measured in several cell lines or cultures. Lane 1) 3-week (immature) mast cell culture; 2) resting mast cell leukemia cell line (HMC-1); 3) activated HMC-1 (activated with ionomycin/PMA). Ribosomal RNA size markers (28s and 18s) are present on the left side of the blot.

FIG. 11 shows a Northern blot in which the expression of a 3.5 kB and a 5.2 kB mRNA species corresponding to SEQ ID NO: 3 were detected in isolated mRNA from various human tissues.

FIG. 12 shows an expression assay in which the intensity of expression of F10317 (MC16; SEQ ID NO: 6) was measured across a panel of cultured mast cells using the Affymetrix GeneChip 42K set. The cultured mast cell age and treatment (resting, activated or reactivated) are indicated. KL starved=mast cells cultured in control medium containing kit ligand for 6 weeks, followed by washing and 7 hours incubation in medium without kit ligand (cytokine). Act=Activation of mature (6 week old) cultured mast cells at time zero by IgE with antigen, and the time periods shown indicate the time elapsed between activation and cell sampling. React=cells were initially activated at six weeks, allowed to recover, activated one week later, allowed to recover, and finally activated at a total of 8 weeks culture time and sampled 3 h after final activation. Urticaria=cells cultured from peripheral blood of a patient with urticaria and allergic hypersensitivity. Cord Blood=mast cells cultured for 6 weeks from umbilical cord blood at the time of birth. Control (6 week old, mature cells)=mast cells cultured in control medium containing kit ligand for 6 weeks.

FIG. 13 shows an electronic Northern assay consisting of normal human tissues in which the intensity of expression of F10317 was measured across a panel of tissues and cell cultures using the Affymetrix human GeneChip 42K chip set. The mean intensity of expression is shown as a horizontal bar that starts at the mean value−standard deviation and stops at the mean+standard deviation.

FIG. 14 shows the relative expression of F10317 (MC16) using quantitative RT-PCR on RNA samples from normal mast cells, mast cells from patients with urticaria or asthma, mature cultured mast cells (unactivated, activated or reactivated, described in FIG. 1 and Example 2 below), various cell lines (KU 812, Jurkat or MO7E), various types of white blood cells (peripheral blood lymphocytes (PBL), monocytes or eosinophils) and cord blood derived mast cells.

FIG. 15 shows a hydrophobicity analysis of the polypeptide of SEQ ID NO: 7 using the methods of Goldman et al. and of Kyle-Doolittle.

FIG. 16 shows a Northern blot in which the expression of a 3.9 kb mRNA species corresponding to SEQ ID NO: 6, F10317 (MC16), was detected in isolated mRNA from various human tissues. Lane M) RNA size markers; 1) spleen; 2) thymus; 3) prostate; 4) testis; 5) uterus; 6) small intestine; 7) colon; 8) leukocytes.

FIG. 17 shows a Northern blot in which the expression of a 3.9 kB and a 4.6 kB mRNA species corresponding to SEQ ID NO: 6, F10317 (MC16), were detected in isolated mRNA from various human tissues. Lane M) RNA size markers; 1) heart; 2) brain; 3) placenta; 4) lung; 5) liver; 6) smooth muscle; 7) kidney; 8) pancreas.

FIG. 18 shows a Northern blot in which the expression of a 3.9 kB mRNA species corresponding to SEQ ID NO: 6, F10317 (MC16), was detected in total RNA from human cultured mast cells (MC). Total RNA from HMC-1 (mast cell leukemia cell line), KU812 (immature basophil cell line) and MO7e (megakaryoblast leukemia cell line) was also examined. Size indicators (in kb) appear on the right side of the blot.

FIG. 19 shows an expression assay in which the level of expression of AA458943 (MC17; SEQ ID NO: 8 or 10) was measured across a panel of cultured mast cells using the Affymetrix GeneChip 42K set. The cultured mast cell age and treatment (resting, activated or reactivated) are indicated. 6 wk, mature=mast cells cultured in control medium containing kit ligand for 6 weeks. KL starved=mast cells cultured in control medium containing kit ligand for 6 weeks, followed by washing and 7 hrs. incubation in medium without kit ligand (cytokine). Act=Activation of mature (6 week old) cultured mast cells at time zero by IgE with antigen, and the time periods shown indicate the time elapsed between activation and cell sampling. React=cells were initially activated at six weeks, allowed to recover, activated one week later, allowed to recover, and finally activated at a total of 8 weeks culture time and sampled 3 h after final activation. Urticaria=cells cultured from peripheral blood of a patient with urticaria and allergic hypersensitivity. Cord Blood=mast cells cultured for 6 weeks from umbilical cord blood at the time of birth.

FIG. 20 shows an electronic Northern assay of normal human tissues in which the intensity of expression of AA458943 (MC17) was measured across a panel of tissues and cell cultures using the Affymetrix human GeneChip 42K chip set. The mean intensity of expression is shown as a horizontal bar that starts at the mean value−standard deviation and stops at the mean+standard deviation.

FIG. 21 shows the relative expression of AA458943 (MC17) using quantitative RT-PCR on RNA samples from normal mast cells, mast cells from patients with urticaria or asthma, mature cultured mast cells (unactivated, activated or reactivated, described in FIG. 19 and Example 4 below), various cell lines (KU 812, Jurkat or Mo7E), various types of white blood cells (peripheral blood lymphocytes (PBL) or monocytes) and cord blood derived mast cells.

FIG. 22 shows a hydrophobicity analysis of the polypeptide of SEQ ID NO: 9 using the methods of Goldman et al. and of Kyle-Doolittle.

FIG. 23 shows a hydrophobicity analysis of the polypeptide of SEQ ID NO: 11 using the methods of Goldman et al. and of Kyle-Doolittle.

FIG. 24 shows a Northern blot, in which the expression level of a 2.7 kb mRNA species corresponding to SEQ ID NO: 8 (clone no. AA458943 (MC17)) was measured in several cell lines or cultures. Lane 1) 3-week (immature) mast cell culture; 2) resting mast cell leukemia cell line (HMC-1); 3) activated HMC-1 (activated with ionomycin/PMA). Ribosomal RNA size markers (28s and 18s) are present on the left side of the blot.

FIG. 25 shows a Northern blot in which the expression of 1.8 kb, 2.7 kb, 3.4 kb and a 4.1 kb mRNA species corresponding to SEQ ID NO: 8 were detected in isolated mRNA from various human tissues. Lane M) RNA size markers; 1) spleen; 2) thymus; 3) prostate; 4) testis; 5) uterus; 6) small intestine; 7) colon; 8) leukocyte.

FIG. 26 shows a western blot of primary cultured mast cells grown from peripheral blood or Jurkat T cells as a negative control. Cell lysate aliquots with (+) or without (−) λ protein phosphatase treatment prior to SDS-PAGE were run side-by-side. Following transfer to membranes, blots were probed with antisera raised to E. coli expressed MC1 protein.

FIG. 27 shows mast cells infected with an adenoviral expression constructs containing the open reading frame of the MC1 cDNA (SEQ ID NO: 1) or no cDNA. Cell viability was analyzed two days after infection by detecting expression of the activation/apoptosis marker annexin V prior to or following mast cell activation with IgE and antigen.

FIG. 28 shows mediator release from mast cells following activation with IgE and antigen is compromised following MC1 overexpression. Mast cells were infected with adenovirus vectors comprising either the MC1 open reading frame of SEQ ID NO: 1 or with control green fluorescent protein (GFP). Release of the degranulation marker β-hexoseaminidase (beta-Hex) and the lipid mediators LTC4 and PGD2 as well as the synthesis of GM-CSF are shown.

### DETAILED DESCRIPTION OF THE PREFERRED EMBODIMENTS

I. General Description

The genes and proteins of the invention were detected in primary human mast cells. These cells were initially isolated as precursor cells from peripheral blood and mature into mast cells over time in culture. The cells respond to antigen in a similar manner as mature mast cells in vivo by degranulation and histamine release. Samples were collected from a patient with urticaria and from umbilical cord blood. Mast cells were purified from these populations and compared to mast cells isolated from normal patients under a variety of conditions. The cells isolated from the urticaria patient are thought to be highly sensitive to stimuli, i.e. have an activated morphology in the absence of a stimulus, during early stages of maturation. As described below, the differential expression of gene species has been studied using these cells.

The present invention is based in part on the identification of a new gene family that is differentially expressed in mast cells following activation or in human patients with allergic hypersensitivity diseases, such as urticaria, compared to non-hypersensitive individuals. This gene family corresponds to the human cDNA of SEQ ID NO: 1, 3, 6, 8, 10 or 12. Genes that encode the human protein of SEQ ID NO: 2, 4, 5, 7, 9, 11 or 13 may also be found in other animal species, particularly mammalian species.

The protein may be used as a marker to detect, diagnose or identify an allergic response in a patient. The protein can also serve as a target for agents that modulate gene expression or activity. For example, agents may be identified that modulate biological processes associated with mast cell function, including mast cell degranulation that leads to urticaria.

The present invention is further based on the development of methods for isolating binding partners that bind to the protein. Additionally, the protein may be used to screen synthetic small molecules and combinatorial or naturally occurring compound libraries to discover novel therapeutics to regulate mast cell function.

II. Specific Embodiments

A. The Proteins Associated with Mast Cell Regranulation and/or Allergic Hypersensitivity

The present invention provides isolated proteins, allelic variants of the proteins, and conservative amino acid substitutions of the proteins. As used herein, the “protein” or “polypeptide” refers, in part, to a protein that has the human amino acid sequence depicted in SEQ ID NO: 2, 4, 5, 7, 9, 11 or 13 or fragments thereof. The terms also refer to naturally occurring allelic variants and proteins that have a slightly different amino acid sequence than that specifically recited above. Allelic variants, though possessing a slightly different amino acid sequence than those recited above, will still have the same or similar biological functions associated with these proteins.

As used herein, the family of proteins related to the human amino acid sequence of SEQ ID NO: 2, 4, 5, 7, 9, 11 or 13 refers in part, to proteins that have been isolated from organisms in addition to humans. The methods used to identify and isolate other members of the family of proteins related to these proteins are described below.

The proteins of the present invention are preferably in isolated form. In some cases, the isolated protein will be processed by the cell from which it is expressed to remove signal sequences, etc. so that the protein is in its processed form. As used herein, a protein is said to be isolated when physical, mechanical or chemical methods are employed to remove the protein from cellular constituents that are normally associated with the protein. A skilled artisan can readily employ standard purification methods to obtain an isolated protein.

The proteins of the present invention further include insertion, deletion or conservative amino acid substitution variants of SEQ ID NO: 2, 4, 5, 7, 9, 11 or 13. As used herein, a conservative variant refers to alterations in the amino acid sequence that do not adversely affect the biological functions of the protein. A substitution, insertion or deletion is said to adversely affect the protein when the altered sequence prevents or disrupts a biological function associated with the protein. For example, the overall charge, structure or hydrophobic/hydrophilic properties of the protein can be altered without adversely affecting a biological activity. Accordingly, the amino acid sequence can be altered, for example to render the peptide more hydrophobic or hydrophilic, without adversely affecting the biological activities of the protein.

Ordinarily, the allelic variants, the conservative substitution variants, and the members of the protein family, will have an amino acid sequence having at least about 35%, 40%, 50%, 60%, 65%, 70% or 75% amino acid sequence identity with the sequence set forth in SEQ ID NO: 2, 4, 5, 7, 9, 11 or 13, more preferably at least about 80%, even more preferably at least about 90%, and most preferably at least about 95% sequence identity. Identity or homology with respect to such sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity (see section B for the relevant parameters). Fusion proteins, or N-terminal, C-terminal or internal extensions, deletions, or insertions into the peptide sequence shall not be construed as affecting homology.

Thus, the proteins of the present invention include molecules having the amino acid sequence disclosed in SEQ ID NO: 2, 4, 5, 7, 9, 11 or 13; fragments thereof having a consecutive sequence of at least about 3, 4, 5, 6, 10, 15, 20, 25, 30, 35 or more amino acid residues of these proteins; amino acid sequence variants wherein one or more amino acid residues has been inserted N- or C-terminal to, or within, the disclosed coding sequence; and amino acid sequence variants of the disclosed sequence, or their fragments as defined above, that have been substituted by at least one residue. Such fragments, also referred to as peptides or polypeptides, may contain antigenic regions, functional regions of the protein identified as regions of the amino acid sequence which correspond to known protein domains, as well as regions of pronounced hydrophilicity. The regions are all easily identifiable by using commonly available protein sequence analysis software such as MacVector (Oxford Molecular).

Contemplated variants further include those containing predetermined mutations by, e.g., homologous recombination, site-directed or PCR mutagenesis, and the corresponding proteins of other animal species, including but not limited to rabbit, mouse, rat, porcine, bovine, ovine, equine and non-human primate species, and the alleles or other naturally occurring variants of the family of proteins; and derivatives wherein the protein has been covalently modified by substitution, chemical, enzymatic, or other appropriate means with a moiety other than a naturally occurring amino acid (for example a detectable moiety such as an enzyme or radioisotope).

The present invention further includes embodiments of the proteins of the invention or variants thereof as described above which have been secreted by a cell. The term “secreted” includes a protein that is transported across or through a membrane, including transport as a result of signal sequences in its amino acid sequence when it is expressed in a suitable host cell. “Secreted” proteins include without limitation proteins secreted wholly (e.g., soluble proteins) or partially (e.g., receptors or membrane bound) from the cell in which they are expressed. “Secreted” proteins also include without limitation proteins that are transported across the membrane of the endoplasmic reticulum. “Secreted” proteins are also intended to include proteins containing non-typical signal sequences and proteins of the invention or variants thereof released from damaged cells.

Where desired, an expression vector may be designed to contain a “signal or leader sequence” which will direct the polypeptide through the membrane of a cell. Such a sequence may be naturally present on the polypeptides of the present invention or provided from heterologous protein sources by recombinant DNA techniques.

As described below, members of the family of proteins can be used: (1) as a diagnostic marker; (2) to identify agents which modulate at least one activity of the protein; (3) to identify binding partners for the protein, (4) as an antigen to raise polyclonal or monoclonal antibodies, and (5) as a therapeutic agent or target.

B. Nucleic Acid Molecules

The present invention further provides nucleic acid molecules that encode the protein having SEQ ID NO: 2, 4, 5, 7, 9, 11 or 13 and the related proteins herein described, preferably in isolated form. As used herein, “nucleic acid” is defined as RNA or DNA or related molecules that encodes a protein or peptide as defined above, is complementary to a nucleic acid sequence encoding such peptides, hybridizes to such a nucleic acid and remains stably bound to it under appropriate stringency conditions, or encodes a polypeptide sharing at least about 35%, 40%, 50%, 60%, 65%, 70% or 75% sequence identity, preferably at least about 80%, more preferably at least about 85%, and even more preferably at least about 90% or 95% or more identity with the peptide sequences. The “nucleic acid molecules” of the invention further include nucleic acid molecules that share at least about 35%, 40%, 50%, 60%, 65%, 70% or 75% sequence identity, preferably at least about 80%, more preferably at least about 85%, and even more preferably at least about 90% or 95% or more identity with the nucleotide sequence of SEQ ID NO: 1, 3, 6, 8, 10 or 12 or the open reading frames defined therein. Specifically contemplated are genomic DNA, cDNA, mRNA and antisense molecules, as well as nucleic acids based on alternative backbones or including alternative bases whether derived from natural sources or synthesized. Such nucleic acids, however, are defined further as being novel and unobvious over any prior art nucleic acid including that which encodes, hybridizes under appropriate stringency conditions, or is complementary to nucleic acid encoding a protein according to the present invention.

Homology or identity at the nucleotide or amino acid sequence level is determined by BLAST (Basic Local Alignment Search Tool) analysis using the algorithm employed by the programs blastp, blastn, blastx, tblastn and tblastx (Altschul, S. F. et al., Nucleic Acids Res 25: 3389-3402 (1997) and Karlin et al., Proc Natl Acad Sci USA 87:2264-2268 (1990), both fully incorporated by reference) which are tailored for sequence similarity searching. The approach used by the BLAST program is to first consider similar segments, with and without gaps, between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al., (1994) (Nature Genetics 6, 119-129) which is fully incorporated by reference. The search parameters for histogram, descriptions, alignments, expect (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix (Henikoff et al., (1992) Proc. Natl. Acad. Sci. USA 89, 10915-10919, fully incorporated by reference), recommended for query sequences over 85 in length (nucleotide bases or amino acids).

For blastn, the scoring matrix is set by the ratios of M (i.e., the reward score for a pair of matching residues) to N (i.e., the penalty score for mismatching residues), wherein the default values for M and N are +5 and −4, respectively. Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

“Stringent conditions” include those that (1) employ low ionic strength and high temperature for washing, for example, 0.015 M NaCl/0.0015 M sodium citrate/0.1% SDS at 50° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is hybridization in 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 0.1% SDS. A skilled artisan can readily determine and vary the stringency conditions appropriately to obtain a clear and detectable hybridization signal. Preferred molecules are those that hybridize under the above conditions to the complement of SEQ ID NO: 1, 3, 6, 8, 10 or 12 and which encode a functional protein. Even more preferred hybridizing molecules are those that hybridize under the above conditions to the complement strand of the open reading frame of SEQ ID NO: 1, 3, 6, 8, 10 or 12.

As used herein, a nucleic acid molecule is said to be “isolated” when the nucleic acid molecule is substantially separated from contaminant nucleic acid molecules encoding other polypeptides.

The present invention further provides fragments of the encoding nucleic acid molecule. As used herein, a fragment of an encoding nucleic acid molecule refers to a small portion of the entire protein coding sequence. The size of the fragment will be determined by the intended use. For example, if the fragment is chosen so as to encode an active portion of the protein, the fragment will need to be large enough to encode the functional region(s) of the protein. For instance, fragments that encode peptides corresponding to predicted antigenic regions may be prepared. If the fragment is to be used as a nucleic acid probe or PCR primer, then the fragment length is chosen so as to obtain a relatively small number of false positives during probing/priming (see the discussion in Section H).

Fragments of the encoding nucleic acid molecules of the present invention (i.e., synthetic oligonucleotides) that are used as probes or specific primers for the polymerase chain reaction (PCR), or to synthesize gene sequences encoding proteins of the invention, can easily be synthesized by chemical techniques, for example, the phosphotriester method of Matteucci et al., (1981) (J. Am. Chem. Soc. 103, 3185-3191) or using automated synthesis methods. In addition, larger DNA segments can readily be prepared by well-known methods, such as synthesis of a group of oligonucleotides that define various modular segments of the gene, followed by ligation of oligonucleotides to build the complete modified gene.

The encoding nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can readily employ any such label to obtain labeled variants of the nucleic acid molecules of the invention.

Modifications to the primary structure of the nucleic acid molecules by deletion, addition, or alteration of the nucleotide sequence can be made without destroying the activity of the encoded proteins. Such substitutions or other alterations result in proteins having an amino acid sequence falling within the contemplated scope of the present invention.

C. Isolation of Other Related Nucleic Acid Molecules

As described above, the identification and characterization of the nucleic acid molecule having SEQ ID NO: 1, 3, 6, 8, 10 or 12 allows a skilled artisan to isolate nucleic acid molecules that encode other members of the protein family in addition to the sequences herein described.

For instance, a skilled artisan can readily use the amino acid sequence of SEQ ID NO: 2, 4, 5, 7, 9, 11 or 13 to generate antibody probes to screen expression libraries prepared from appropriate cells. Typically, polyclonal antiserum from mammals such as rabbits immunized with the purified protein (as described below) or monoclonal antibodies can be used to probe a mammalian cDNA or genomic expression library, such as lambda gtl1 library, to obtain the appropriate coding sequence for other members of the protein family. The cloned cDNA sequence can be expressed as a fusion protein, expressed directly using its own control sequences, or expressed by constructions using control sequences appropriate to the particular host used for expression of the enzyme.

Alternatively, a portion of the coding sequence herein described can be synthesized and used as a probe to retrieve DNA encoding a member of the protein family from any mammalian organism. Oligomers containing approximately 18-20 nucleotides (encoding about a 6-7 amino acid stretch) are prepared and used to screen genomic DNA or cDNA libraries to obtain hybridization under stringent conditions or conditions of sufficient stringency to eliminate an undue level of false positives.

Additionally, pairs of oligonucleotide primers can be prepared for use in a polymerase chain reaction (PCR) to selectively clone an encoding nucleic acid molecule. A PCR denature/anneal/extend cycle for using such PCR primers is well known in the art and can readily be adapted for use in isolating other encoding nucleic acid molecules.

Nucleic acid molecules encoding other members of the protein family may also be identified in existing genomic or other sequence information using any available computational method, including but not limited to: PSI-BLAST (Altschul, et al. (1997) Nucleic Acids Res. 25:3389-3402); PHI-BLAST (Zhang, et al. (1998), Nucleic Acids Res. 26:3986-3990), 3D-PSSM (Kelly et al. (2000) J. Mol. Biol. 299(2): 499-520); and other computational analysis methods (Shi et al. (1999) Biochem. Biophys. Res. Commun. 262(1):132-8 and Matsunami et. al (2000) Nature 404(6778):601-4.

D. rDNA molecules Containing a Nucleic Acid Molecule

The present invention further provides recombinant DNA molecules (rDNAs) that contain a coding sequence. As used herein, a rDNA molecule is a DNA molecule that has been subjected to molecular manipulation in situ. Methods for generating rDNA molecules are well known in the art, for example, see Sambrook et al., (1989) Molecular Cloning—A Laboratory Manual, Cold Spring Harbor Laboratory Press. In the preferred rDNA molecules, a coding DNA sequence is operably linked to expression control sequences and/or vector sequences.

The choice of vector and/or expression control sequences to which one of the protein family encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 available from BioRad Laboratories, (Richmond, Calif.), pPL and pKK223 available from Pharmacia (Piscataway, N.J.).

Expression vectors compatible with eukaryotic cells, preferably those compatible with vertebrate cells, can also be used to form rDNA molecules that contain a coding sequence. Eukaryotic cell expression vectors, including viral vectors, are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies, Inc.), pTDT1 (ATCC, #31255), the vector pCDM8 described herein, and the like eukaryotic expression vectors.

Eukaryotic cell expression vectors used to construct the rDNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene. (Southern et al., (1982) J. Mol. Anal. Genet. 1, 327-341) Alternatively, the selectable marker can be present on a separate plasmid, and the two vectors are introduced by co-transfection of the host cell, and selected by culturing in the appropriate drug for the selectable marker.

E. Host Cells Containing an Exogenously Supplied Coding Nucleic Acid Molecule

The present invention further provides host cells transformed with a nucleic acid molecule that encodes a protein of the present invention. The host cell can be either prokaryotic or eukaryotic. Eukaryotic cells useful for expression of a protein of the invention are not limited, so long as the cell line is compatible with cell culture methods and compatible with the propagation of the expression vector and expression of the gene product. Preferred eukaryotic host cells include, but are not limited to, yeast, insect and mammalian cells, preferably vertebrate cells such as those from a mouse, rat, monkey or human cell line. Preferred eukaryotic host cells include Chinese hamster ovary (CHO) cells available from the ATCC as CCL61, NIH Swiss mouse embryo cells (NIH/3T3) available from the ATCC as CRL 1658, baby hamster kidney cells (BHK), and the like eukaryotic tissue culture cell lines.

Any prokaryotic host can be used to express a rDNA molecule encoding a protein of the invention. The preferred prokaryotic host is E. coli.

Transformation of appropriate cell hosts with a rDNA molecule of the present invention is accomplished by well-known methods that typically depend on the type of vector used and host system employed. With regard to transformation of prokaryotic host cells, electroporation and salt treatment methods are typically employed, see, for example, Cohen et al., (1972) Proc. Natl. Acad. Sci. USA 69, 2110; and Sambrook et al., (1989) Molecular Cloning—A Laboratory Manual, Cold Spring Harbor Laboratory Press. With regard to transformation of vertebrate cells with vectors containing rDNAs, electroporation, cationic lipid or salt treatment methods are typically employed, see, for example, Graham et al., (1973) Virol. 52, 456; Wigler et al., (1979) Proc. Natl. Acad. Sci. USA 76, 1373-1376.

Successfully transformed cells, i.e., cells that contain a rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern, J. Mol. Biol. 98:503, 1975, or Berent et al., (1985) Biotech. 3, 208 or the proteins produced from the cell assayed via an immunological method.

F. Production of Recombinant Proteins using a rDNA Molecule

The present invention further provides methods for producing a protein of the invention using nucleic acid molecules herein described. In general terms, the production of a recombinant form of a protein typically involves the following steps:

A nucleic acid molecule is first obtained that encodes a protein of the invention, such as a nucleic acid molecule comprising, consisting essentially of or consisting of SEQ ID NO: 1, 3, 6, 8, 10 or 12, nucleotides 25-429 (or 432) of SEQ ID NO: 1, nucleotides 294-743 (or 746) or 1238-2215 (or 2218) of SEQ ID NO: 3, nucleotides 377-1948 (or 1951) of SEQ ID NO: 6, nucleotides 162-632 (or 635) of SEQ ID NO: 8 or 373-648 (or 651) of SEQ ID NO: 10. SEQ ID NOS: 8 and 10 are identical at the nucleotide level, but have different open reading frames, as indicated. If the encoding sequence is uninterrupted by introns, as are these open reading frames, it is directly suitable for expression in any host.

The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce recombinant protein.

G. Methods to Identify Binding Partners

Another embodiment of the present invention provides methods of isolating and identifying binding partners of proteins of the invention. In general, a protein of the invention is mixed with a potential binding partner or an extract or fraction of a cell under conditions that allow the association of potential binding partners with the protein of the invention. After mixing, peptides, polypeptides, proteins or other molecules that have become associated with a protein of the invention are separated from the mixture. The binding partner that bound to the protein of the invention can then be removed and further analyzed. To identify and isolate a binding partner, the entire protein, for instance a protein comprising the entire amino acid sequence of SEQ ID NO: 2, 4, 5, 7, 9, 11 or 13 can be used. Alternatively, a fragment of the protein can be used.

As used herein, a cellular extract refers to a preparation or fraction that is made from a lysed or disrupted cell. The preferred source of cellular extracts will be cells derived from human skin tissue or the human respiratory tract or cells derived from a biopsy sample of human lung tissue in patients with allergic hypersensitivity. Alternatively, cellular extracts may be prepared from normal tissue or available cell lines, particularly granulocytic cell lines.

A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with the protein of the invention under conditions in which association of the protein with the binding partner can occur. A variety of conditions can be used, the most preferred being conditions that closely resemble conditions found in the cytoplasm of a human cell. Features such as osmolarity, pH, temperature, and the concentration of cellular extract used, can be varied to optimize the association of the protein with the binding partner.

After mixing under appropriate conditions, the bound complex is separated from the mixture. A variety of techniques can be utilized to separate the mixture. For example, antibodies specific to a protein of the invention can be used to immunoprecipitate the binding partner complex. Alternatively, standard chemical separation techniques such as chromatography and density/sediment centrifugation can be used.

After removal of non-associated cellular constituents found in the extract, the binding partner can be dissociated from the complex using conventional methods. For example, dissociation can be accomplished by altering the salt concentration or pH of the mixture.

To aid in separating associated binding partner pairs from the mixed extract, the protein of the invention can be immobilized on a solid support. For example, the protein can be attached to a nitrocellulose matrix or acrylic beads. Attachment of the protein to a solid support aids in separating peptide/binding partner pairs from other constituents found in the extract. The identified binding partners can be either a single protein or a complex made up of two or more proteins. Alternatively, binding partners may be identified using a Far-Western assay according to the procedures of Takayama et al., (1997) Methods Mol. Biol. 69:171-184 or Sauder et al., (1996) J. Gen. Virol. 77:991-996 or identified through the use of epitope tagged proteins or GST fusion proteins.

Alternatively, the nucleic acid molecules of the invention can be used in a yeast two-hybrid system. The yeast two-hybrid system has been used to identify other protein partner pairs and can readily be adapted to employ the nucleic acid molecules herein described.

H. Methods to Identify Agents that Modulate the Expression of a Nucleic Acid Encoding the Gene Associated with Mast Cell Degranulation or Allergic Hypersensitivity

Another embodiment of the present invention provides methods for identifying agents that modulate the expression of a nucleic acid encoding a protein of the invention such as a protein having the amino acid sequence of SEQ ID NO: 2, 4, 5, 7, 9, 11 or 13. Such assays may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, cell lines that contain reporter gene fusions between the open reading frame defined by nucleotides 25-429 of SEQ ID NO: 1, 294-743 or 1238-2215 of SEQ ID NO: 3, 377-1948 of SEQ ID NO: 6, 162-632 of SEQ ID NO: 8 or 373-648 of SEQ ID NO: 10 and/or the 5′ and/or 3′ regulatory elements and any assayable fusion partner may be prepared. Numerous assayable fusion partners are known and readily available including the firefly luciferase gene and the gene encoding chloramphenicol acetyltransferase (Alam et al., (1990) Anal. Biochem. 188, 245-254). Cell lines containing the reporter gene fusions are then exposed to the agent to be tested under appropriate conditions and time. Differential expression of the reporter gene between samples exposed to the agent and control samples identifies agents that modulate the expression of a nucleic acid of the invention.

Additional assay formats may be used to monitor the ability of the agent to modulate the expression of a nucleic acid encoding a protein of the invention, such as the protein having SEQ ID NO: 2, 4, 5, 7, 9, 11 or 13. For instance, mRNA expression may be monitored directly by hybridization to the nucleic acids of the invention. Cell lines are exposed to the agent to be tested under appropriate conditions and time and total RNA or mRNA is isolated by standard procedures such those disclosed in Sambrook et al., (1989) Molecular Cloning—A Laboratory Manual, Cold Spring Harbor Laboratory Press).

Probes to detect differences in RNA expression levels between cells exposed to the agent and control cells may be prepared from the nucleic acids of the invention. It is preferable, but not necessary, to design probes that hybridize only with target nucleic acids under conditions of high stringency. Only highly complementary nucleic acid hybrids form under conditions of high stringency. Accordingly, the stringency of the assay conditions determines the amount of complementarity that should exist between two nucleic acid strands in order to form a hybrid. Stringency should be chosen to maximize the difference in stability between the probe:target hybrid and probe:non-target hybrids.

Probes may be designed from the nucleic acids of the invention through methods known in the art. For instance, the G+C content of the probe and the probe length can affect probe binding to its target sequence. Methods to optimize probe specificity are commonly available in Sambrook et al., (1989) Molecular Cloning—A Laboratory Manual, Cold Spring Harbor Laboratory Press) or Ausubel et al., (1995) Current Protocols in Molecular Biology, Greene Publishing Co.

Hybridization conditions are modified using known methods, such as those described by Sambrook et al. and Ausubel et al. as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The wafer can then be exposed to total cellular RNA or polyA RNA from a sample under conditions in which the affixed sequences will specifically hybridize. Such solid supports and hybridization methods are widely available, for example, those disclosed by Beattie, (1995) WO 95/11755. By examining for the ability of a given probe to specifically hybridize to an RNA sample from an untreated cell population and from a cell population exposed to the agent, agents which up or down regulate the expression of a nucleic acid encoding the protein having the sequence of SEQ ID NO: 2, 4, 5, 7, 9, 11 or 13 are identified.

Hybridization for qualitative and quantitative analysis of mRNAs may also be carried out by using a RNase Protection Assay (i.e., RPA, see Ma et al. (1996) Methods 10, 273-238). Briefly, an expression vehicle comprising cDNA encoding the gene product and a phage specific DNA dependent RNA polymerase promoter (e.g., T7, T3 or SP6 RNA polymerase) is linearized at the 3′ end of the cDNA molecule, downstream from the phage promoter, wherein such a linearized molecule is subsequently used as a template for synthesis of a labeled antisense transcript of the cDNA by in vitro transcription. The labeled transcript is then hybridized to a mixture of isolated RNA (i.e., total or fractionated mRNA) by incubation at 45° C. overnight in a buffer comprising 80% formamide, 40 mM Pipes, pH 6.4, 0.4 M NaCl and 1 mM EDTA. The resulting hybrids are then digested in a buffer comprising 40 μg/ml ribonuclease A and 2 μg/ml ribonuclease. After deactivation and extraction of extraneous proteins, the samples are loaded onto urea/polyacrylamide gels for analysis.

In another assay format, cells or cell lines are first identified which express the gene products of the invention physiologically. Cell and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Further, such cells or cell lines would be transduced or transfected with an expression vehicle (e.g., a plasmid or viral vector) construct comprising an operable non-translated 5′-promoter containing end of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag or other detectable marker. Such a process is well known in the art (see Sambrook et al., (1989)).

Cells or cell lines transduced or transfected as outlined above are then contacted with agents under appropriate conditions; for example, the agent in a pharmaceutically acceptable excipient is contacted with cells in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells will be disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample will be compared with a control sample where only the excipient is contacted with the cells and an increase or decrease in the immunologically generated signal from the “agent-contacted” sample compared to the control will be used to distinguish the effectiveness of the agent.

I. Methods to Identify Agents that Modulate the Level of or at Least One Activity of the Proteins Associated with Mast Cell Degranulation and/or Allergic Hypersensitivity

Another embodiment of the present invention provides methods for identifying agents that modulate at least one activity of a protein of the invention such as the protein having the amino acid sequence of SEQ ID NO: 2, 4, 5, 7, 9, 11 or 13. Such methods or assays may utilize any means of monitoring or detecting the desired activity.

In one format, the relative amounts of a protein of the invention between a cell population that has been exposed to the agent to be tested compared to an unexposed control cell population may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe.

Antibody probes are prepared by immunizing suitable mammalian hosts in appropriate immunization protocols using the peptides, polypeptides or proteins of the invention if they are of sufficient length, or, if desired, or if required to enhance immunogenicity, conjugated to suitable carriers. Methods for preparing immunogenic conjugates with carriers such as BSA, KLH, or other carrier proteins are well known in the art. In some circumstances, direct conjugation using, for example, carbodiimide reagents may be effective; in other instances linking reagents such as those supplied by Pierce Chemical Co. (Rockford, Ill.), may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

While the polyclonal antisera produced in this way may be satisfactory for some applications, for pharmaceutical compositions, use of monoclonal preparations is preferred. Immortalized cell lines which secrete the desired monoclonal antibodies may be prepared using the standard method of Kohler and Milstein (Nature (1975) 256:495-497) or modifications which effect immortalization of lymphocytes or spleen cells, as is generally known. The immortalized cell lines secreting the desired antibodies are screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies are then recovered from the culture supernatant or from the ascites supernatant. Fragments of the monoclonal antibodies or the polyclonal antisera that contain the immunologically significant portion can be used as antagonists, as well as the intact antibodies. Use of immunologically reactive antibody fragments, such as the Fab, Fab′, of F(ab′)₂ fragments is often preferable, especially in a therapeutic context, as these fragments are generally less immunogenic than the whole immunoglobulin.

The antibodies or fragments may also be produced, using current technology, by recombinant means. Antibody regions that bind specifically to the desired regions of the protein can also be produced in the context of chimeras with multiple species origin, such as humanized antibodies. The antibodies may be used in any of the methods described herein, may be used as diagnostic agents or may be used as therapeutic agents.

Agents that are assayed in the above method can be randomly selected or rationally selected or designed. As used herein, an agent is said to be randomly selected when the agent is chosen randomly without considering the specific sequences involved in the association of a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agents action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates. Dominant negative proteins, DNAs encoding these proteins, antibodies to these proteins, peptide fragments of these proteins or mimics of these proteins may be introduced into cells to affect function. “Mimic” used herein refers to the modification of a region or several regions of a peptide molecule to provide a structure chemically different from the parent peptide but topographically and functionally similar to the parent peptide (see Grant G A. in: Meyers (ed.) Molecular Biology and Biotechnology (New York, VCH Publishers, 1995), pp. 659-664). A skilled artisan can readily recognize that there is no limit as to the structural nature of the agents of the present invention.

The peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention are antibodies immunoreactive with critical positions of proteins of the invention. Antibody agents are obtained by immunization of suitable mammalian subjects with peptides, containing as antigenic regions, those portions of the protein intended to be targeted by the antibodies.

J. Uses for Agents that Modulate at Least One Activity of the Proteins.

As provided in the Examples, the proteins and nucleic acids of the invention, such as the protein having the amino acid sequence of SEQ ID NO: 2, 4, 5, 7, 9, 11 or 13, are differentially expressed during mast cell degranulation and/or in allergic hypersensitivity disease states. Agents that modulate or up-or-down-regulate the expression of the protein or agents, such as agonists or antagonists of at least one activity of the protein, may be used to modulate biological and pathologic processes associated with the protein's function and activity.

As used herein, a subject can be any mammal, so long as the mammal is in need of modulation of a pathological or biological process mediated by a protein of the invention. The term mammal is defined as an individual belonging to the class Mammalia. The invention is particularly useful in the treatment of human subjects.

Pathological processes refer to a category of biological processes that produce a deleterious effect. For example, expression of a protein of the invention may be associated with allergic hypersensitivity. As used herein, an agent is said to modulate a pathological process when the agent reduces the degree or severity of the process. For instance, allergic hypersensitivity may be prevented or disease progression modulated by the administration of agents which up- or down-regulate or modulate in some way the expression or at least one activity of a protein of the invention.

The agents of the present invention can be provided alone, or in combination with other agents that modulate a particular pathological process. For example, an agent of the present invention can be administered in combination with other known drugs. As used herein, two agents are said to be administered in combination when the two agents are administered simultaneously or are administered independently in a fashion such that the agents will act at the same time.

The agents of the present invention can be administered via parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired.

The present invention further provides compositions containing one or more agents that modulate expression or at least one activity of a protein of the invention. While individual needs vary, determination of optimal ranges of effective amounts of each component is within the skill of the art. Typical dosages comprise 0.1 to 100 μg/kg body wt. The preferred dosages comprise 0.1 to 10 μg/kg body wt. The most preferred dosages comprise 0.1 to 1 μg/kg body wt.

In addition to the pharmacologically active agent, the compositions of the present invention may contain suitable pharmaceutically acceptable carriers comprising excipients and auxiliaries that facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

In practicing the methods of this invention, the compounds of this invention may be used alone or in combination, or in combination with other therapeutic or diagnostic agents. In certain preferred embodiments, the compounds of this invention may be coadministered along with other compounds typically prescribed for these conditions according to generally accepted medical practice such as antihistamines. The compounds of this invention can be utilized in vivo, ordinarily in mammals, such as humans, sheep, horses, cattle, pigs, dogs, cats, rats and mice, or in vitro.

K. Transgenic Animals

Transgenic animals containing mutant, knock-out or modified genes corresponding to the cDNA sequence of SEQ ID NO: 1, 3, 6, 8, 10 or 12, or the open reading frame encoding the polypeptide sequence of SEQ ID NO: 2, 4, 5, 7, 9, 11 or 13, or fragments thereof having a consecutive sequence of at least about 3, 4, 5, 6, 10, 15, 20, 25, 30, 35 or more amino acid residues, are also included in the invention. Transgenic animals are genetically modified animals into which recombinant, exogenous or cloned genetic material has been experimentally transferred. Such genetic material is often referred to as a “transgene.” The nucleic acid sequence of the transgene, in this case a form of SEQ ID NO: 1, 3, 6, 8, 10 or 12 may be integrated either at a locus of a genome where that particular nucleic acid sequence is not otherwise normally found or at the normal locus for the transgene. The transgene may consist of nucleic acid sequences derived from the genome of the same species or of a different species than the species of the target animal.

The term “germ cell line transgenic animal” refers to a transgenic animal in which the genetic alteration or genetic information was introduced into a germ line cell, thereby conferring the ability of the transgenic animal to transfer the genetic information to offspring. If such offspring in fact possess some or all of that alteration or genetic information, then they too are transgenic animals.

The alteration or genetic information may be foreign to the species of animal to which the recipient belongs, foreign only to the particular individual recipient, or may be genetic information already possessed by the recipient. In the last case, the altered or introduced gene may be expressed differently than the native gene.

Transgenic animals can be produced by a variety of different methods including transfection, electroporation, microinjection, gene targeting in embryonic stem cells and recombinant viral and retroviral infection (see, e.g., U.S. Pat. No. 4,736,866; U.S. Pat. No. 5,602,307; Mullins et al., (1993) Hypertension 22, 630-633; Brenin et al., (1997) Surg. Oncol. 6, 99-110; Tuan (1997) Recombinant Gene Expression Protocols, Methods in Molecular Biology, Humana Press).

A number of recombinant or transgenic mice have been produced, including those which express an activated oncogene sequence (U.S. Pat. No. 4,736,866); express simian SV40 T-antigen (U.S. Pat. No. 5,728,915); lack the expression of interferon regulatory factor 1 (IRF-1) (U.S. Pat. No. 5,731,490); exhibit dopaminergic dysfunction (U.S. Pat. No. 5,723,719); express at least one human gene which participates in blood pressure control (U.S. Pat. No. 5,731,489); display greater similarity to the conditions existing in naturally occurring Alzheimer's disease (U.S. Pat. No. 5,720,936); have a reduced capacity to mediate cellular adhesion (U.S. Pat. No. 5,602,307); possess a bovine growth hormone gene (Clutter et al., (1996) Genetics 143, 1753-1760); or, are capable of generating a filly human antibody response (McCarthy (1997) Lancet 349, 405).

While mice and rats remain the animals of choice for most transgenic experimentation, in some instances it is preferable or even necessary to use alternative animal species. Transgenic procedures have been successfully utilized in a variety of non-murine animals, including sheep, goats, pigs, dogs, cats, monkeys, chimpanzees, hamsters, rabbits, cows and guinea pigs (see, e.g., Kim et al., (1997) Mol. Reprod. Dev. 46, 515-526; Houdebine (1995) Reprod. Nutr. Dev. 35, 609-617; Petters (1994) Reprod. Fertil. Dev. 6, 643-645; Schnieke et al., (1997) Science 278, 2130-2133; and Amoah (1997) J. Animal Science 75, 578-585).

The method of introduction of nucleic acid fragments into recombination competent mammalian cells can be by any method that favors co-transformation of multiple nucleic acid molecules. Detailed procedures for producing transgenic animals are readily available to one skilled in the art, including the disclosures in U.S. Pat. No. 5,489,743 and U.S. Pat. No. 5,602,307.

L. Diagnostic Methods and Agents

The genes and proteins of the invention may be used to diagnose or monitor allergic hypersensitivity diseases, seasonal rhinitis, asthma, atopic dermatitis and amstocytosis or to track disease progression. Some genes and proteins of the invention are differentially expressed in skin tissue from patients with allergic hypersensitivity compared to normal skin tissue, the genes and proteins of the invention. One means of diagnosing allergic hypersensitivity using the nucleic acid molecules or proteins of the invention involves obtaining cells from skin, lung or respiratory tract tissue from living subjects. When possible, mucosal secretions, urine, blood or peripheral lymphocyte samples may be used as the tissue sample in the assay.

The use of molecular biological tools has become routine in forensic technology. For example, nucleic acid probes may be used to determine the expression of a nucleic acid molecule comprising all or at least part of the sequences of SEQ ID NO: 1, 3, 6, 8, 10 or 12 in forensic/pathology specimens. Further, nucleic acid assays may be carried out by any means of conducting a transcriptional profiling analysis. In addition to nucleic acid analysis, forensic methods of the invention may target the proteins of the invention, particularly a protein comprising SEQ ID NO: 2, 4, 5, 7, 9, 11 or 13, to determine up or down regulation of the genes (Shiverick et al., (1975) Biochim Biophys Acta 393, 124-133).

Methods of the invention may involve treatment of tissues with collagenases or other proteases to make the tissue amenable to cell lysis (Semenov et al., (1987) Biull Eksp Biol Med 104, 113-116). Further, it is possible to obtain biopsy samples from different regions of the skin, respiratory tract or lungs for analysis.

Assays to detect nucleic acid or protein molecules of the invention may be in any available format. Typical assays for nucleic acid molecules include hybridization or PCR based formats. Typical assays for the detection of proteins, polypeptides or peptides of the invention include the use of antibody probes in any available format such as in situ binding assays, etc. See Harlow & Lane, (1988) Antibodies—A Laboratory Manual, Cold Spring Harbor Laboratory Press. In preferred embodiments, assays are carried-out with appropriate controls.

The above methods may also be used in other diagnostic protocols, including protocols and methods to detect disease states in other tissues or organs, for example the tissues in which gene expression is detected.

M. Databases and Computer-Readable Formats

In one application of this embodiment, nucleotide or protein sequence of the present invention can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM, CD-R, CD-R/W or DVD; electrical storage media such as RAM and ROM; and hybrids of these categories such as magnetic/optical storage media. A skilled artisan can readily appreciate how any of the presently known computer readable media can be used to create a manufacture comprising computer readable medium having recorded thereon a nucleotide or protein sequence of the present invention. As used herein, “recorded” refers to a process for storing information on computer readable medium. A skilled artisan can readily adopt any of the presently known methods for recording information on computer readable medium to generate manufactures comprising a nucleotide or protein sequence information of the present invention.

A variety of data storage structures are available to a skilled artisan for creating a computer readable medium having recorded thereon a nucleotide or protein sequence of the present invention. The choice of the data storage structure will generally be based on the means chosen to access the stored information. In addition, a variety of data processor programs and formats can be used to store the nucleotide or protein sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. A skilled artisan can readily adapt any number of data processor structuring formats (i.e., text file or database) in order to obtain computer readable medium having recorded thereon a nucleotide or protein sequence information of the present invention.

By providing any of the nucleotide sequences SEQ ID NO: 1, 3, 6, 8, 10, 12 or a fragment thereof; or a nucleotide sequence which encodes the protein or polypeptide sequence of SEQ ID NO: 2, 4, 5, 7, 9, 11, 13 or a fragment thereof, or any one of the protein sequences SEQ ID NO: 2, 4, 5, 7, 9, 11, 13 or a fragment thereof, or a nucleotide sequence at least 95% identical to any of the nucleotide sequences of SEQ ID NO: 1, 3, 6, 8, 10 or 12 or at least 95% identical to any nucleotide sequence which encodes the protein or polypeptide sequence of SEQ ID NO: 2, 4, 5, 7, 9, 11, 13 in computer readable form, a skilled artisan can routinely access the sequence information through a user interface for a variety of purposes. Computer software is publicly available which allows a skilled artisan to access sequence information provided in a computer readable medium. For example, computer readable formats of the nucleic acid sequences of the present invention can be used to implement the BLAST (Altschul et al, J. Mol. Biol. 215:403-410 (1990)) and BLAZE (Brutlag et al., Comp. Chem.; 17:203-207 (1993)) search algorithms.

As used herein, “a computer-based system” refers to the hardware means, software means, user interface and data storage means used to analyze the nucleotide or protein sequence information of the present invention. The minimum hardware means of the computer-based systems of the present invention comprises a central processing unit (CPU), input means, output means, and data storage means. A skilled artisan can readily appreciate that any one of the currently available computer-based systems are suitable for use in the present invention. As stated above, the computer-based systems of the present invention comprise a data storage means having stored therein a nucleotide or protein sequence of the present invention and the necessary hardware means and software means for supporting and implementing a search means. As used herein, “data storage means” refers to memory which can store nucleotide sequence information of the present invention, or a memory access means which can access manufactures having recorded thereon the nucleotide sequence information of the present invention.

As used herein, “search means” refers to one or more programs that are implemented on the computer-based system to compare a target sequence or target structural motif with the sequence information stored within the data storage means. Search means are used to identify fragments or regions of a known sequence which match a particular target sequence or target motif. A variety of known algorithms are disclosed publicly and a variety of commercially available software for conducting search means are and can be used in the computer-based systems of the present invention. Examples of such software includes, but is not limited to Smith-Waterman, MacPattern (EMBL), BLASTN and BLASTA (NPOLYPEPTIDEIA). A skilled artisan can readily recognize that any one of the available algorithms or implementing software packages for conducting homology searches can be adapted for use in the present computer-based systems. As used herein, a “target sequence” can be any nucleic acid or amino acid to sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 300 amino acids, more preferably from about 30 to 100 nucleotide residues. However, it is well recognized that searches for commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen based on a three-dimensional configuration which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

N. Mast Cell Stimulating or Suppressing Activity

A protein of the present invention, or a biologically active fragment thereof, may also exhibit stimulatory or suppressive activity on mast cells. By “biologically active fragment” it is meant that said fragment is capable of mediating the same or similar biological activity as the full-length protein from which the fragment is derived, including instances where the activity of the fragment may be diminished or enhanced versus that of the full-length protein. A polynucleotide of the invention can encode a polypeptide exhibiting such activities. A protein may be useful in the treatment or prevention of various diseases or conditions associated with mast cell activity or lack thereof, e.g., in regulating (up or down) growth, maturation, proliferation and activation of mast cells. These diseases or conditions may be genetic or be caused by viral as well as bacterial or fungal infections, or may result from autoimmune disorders.

Using the proteins of the invention it may also be possible to modulate mast cell activities, in a number of ways. Down regulation may be in the form of inhibiting or blocking a mast cell function already in progress or may involve preventing the induction of a mast cell function. The functions of activated mast cells may be inhibited by suppressing mast cell responses or by inducing specific non-responsiveness in mast cells, or both.

Treatment of a subject with a protein of the present invention may be useful for down regulating or preventing one or more mast cell functions, e.g., preventing high level synthesis or release by activated mast cells of mast cell products such as histamine, heparin, eosinophil chemotactic factor, proteases and newly formed lipid mediators, such as leukotrienes and prostaglandins, for example. Such down regulation of mast cell synthesis or release of mediators may be useful for the treatment of various diseases or conditions associated with mast cell activity, such as allergic hypersensitivity, seasonal rhinitis, asthma, urticaria, atopic dermatitis, mastocytosis, for example.

Upregulation of growth, maturation, proliferation and/or activation, as a means of up regulating mast cell responses, may also be useful in therapy. Upregulation of responses may be in the form of enhancing an existing response or eliciting an initial response. For example, enhancing mast cell response may be useful in cases of viral infection, including systemic viral diseases such as influenza, the common cold, and encephalitis.

O. Uses for Agents that Modulate the Expression or at Least One Activity of the Proteins of the Invention

The proteins and nucleic acids of the invention, such as one or more of the proteins having the amino acid sequence of SEQ ID NO: 2, 4 5, 7, 9, 11 or 13, are differentially expressed in mast cells during maturation or in association with a disease process or condition which involves mast cell activity or lack thereof. Said disease process or condition may be selected from, but is not limited to, allergic hypersensitivity, seasonal rhinitis, asthma, urticaria, atopic dermatitis or mastocytosis, for example. Agents that up- or down-regulate or modulate the expression of the protein or at least one activity of the protein, such as agonists or antagonists, of may be used to modulate biological and pathologic processes associated with the protein's function and activity.

As used herein, a subject can be any mammal, so long as the mammal is in need of modulation of a pathological or biological process mediated by a protein of the invention. The term “mammal” is defined as an individual belonging to the class Mammalia. The invention is particularly useful in the treatment of human subjects.

Pathological processes refer to a category of biological processes that produce a deleterious effect. For example, expression of a protein of the invention may be associated with mast cell maturation or regulation. As used herein, an agent is said to modulate a pathological process when the agent reduces the degree or severity of the process. For instance, a disease process or condition which involves mast cell activity or lack thereof may be prevented or modulated by the administration of agents which up- or down-regulate or modulate in some way the expression or at least one activity of a protein of the invention.

The agents of the present invention can be provided alone, or in combination with other agents that modulate a particular pathological process. For example, an agent of the present invention can be administered in combination with other known drugs. As used herein, two agents are said to be administered in combination when the two agents are administered simultaneously or are administered independently in a fashion such that the agents will act at the same time.

The agents of the present invention can be administered via parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired.

The present invention further provides compositions containing one or more agents which modulate expression or at least one activity of a protein of the invention. While individual needs vary, determination of optimal ranges of effective amounts of each component is within the skill of the art. Typical dosages comprise 0.1 to 100 μg/kg body wt. The preferred dosages comprise 0.1 to 10 μg/kg body wt. The most preferred dosages comprise 0.1 to 1 μg/kg body wt.

In addition to the pharmacologically active agent, the compositions of the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

In practicing the methods of this invention, the compounds of this invention may be used alone or in combination, or in combination with other therapeutic or diagnostic agents. In certain preferred embodiments, the compounds of this invention may be coadministered along with other compounds typically prescribed for these conditions according to generally accepted medical practice. The compounds of this invention can be utilized in vivo, ordinarily in mammals, such as humans, sheep, horses, cattle, pigs, dogs, cats, rats and mice, or in vitro.

Without further description, it is believed that one of ordinary skill in the art can, using the preceding description and the following illustrative examples, make and utilize the compounds of the present invention and practice the claimed methods. The following working examples therefore, specifically point out preferred embodiments of the present invention, and are not to be construed as limiting in any way the remainder of the disclosure.

### EXAMPLES

### Example 1

Identification of Differentially Expressed mRNA in Isolated Mast Cells

Primary human mast cells, initially isolated as precursor cells (CD34+ progenitor cells) from peripheral blood, were produced by in vitro cell culture. The cells possess many of the functional properties of mature tissue-derived mast cells, including a dependence on growth factors for survival and function and an IgE-receptor-mediated degranulation. Cell samples for culture were collected from normal donors, as well as from a patient with urticaria, and from umbilical cord blood. Mast cell progenitors were purified from these donor populations, cultured 2-8 weeks and underwent various treatments. Mast cells from normal donors were collected at various stages of maturation, time points after activation, and after multiple cycles of activation. The cells isolated from the urticaria patient appeared to be partially activated in the absence of exogenous antigen stimulation. The cells that have undergone multiple rounds of activation were used to model mast cells from patients with allergic hypersensitivity that be chronically exposed to environmental allergens. The differential expression of gene species has been studied using these different cell populations.

Mast cells were cultured from progenitors with modifications of methods described by Saito et al., J Immunol, 157:343-351, 1996. Mast cells were cultured for 2-8 weeks in medium containing Kit Ligand and conditioned medium from the MCCM cell line. The medium also contained 30% fetal bovine serum that had been treated with activated charcoal. For activation samples, mast cells were first sensitized with IgE specific to tri-nitrophenol (TNP), from the murine hybridoma IgELA2, ATCC #TIB-142. Cells were then washed and activated with TNP-BSA. Microarray sample preparation was conducted with minor modifications, following the protocols set forth in the Affymetrix GeneChip® Expression Analysis Manual. Total RNA from cells was extracted with the acid phenol method (Chomczynski and Sacchi, 1987). The RNA samples were subsequently treated with heparinase (Sigma), as heparin co-purifies with RNA and inhibits the reverse transcriptase reaction. mRNA was isolated using the Oligotex mRNA Midi kit® (Qiagen) followed by ethanol precipitation. Double stranded cDNA was generated from mRNA using the SuperScript Choice® system (GibcoBRL). First strand cDNA synthesis was primed with a T7-(dT24) oligonucleotide. The cDNA was phenol-chloroform extracted and ethanol precipitated to a final concentration of 1 μg/μl. From 2 μg of cDNA, cRNA was synthesized using Ambion's T7 MegaScript in vitro Transcription Kit®.

To biotin label the cRNA, nucleotides Bio-11-CTP and Bio-16-UTP (Enzo Diagnostics) were added into the reaction. Following a 37° C. incubation for six hours, impurities were removed from the labeled cRNA following the RNAeasy Mini kit protocol (Qiagen). cRNA was fragmented (5′ fragmentation buffer consisting of 200 mM Tris-acetate (pH 8.1), 500 mM KOAc, 150 mM MgOAc) for thirty-five minutes at 94° C. Following the Affymetrix protocol, 55 μg of fragmented cRNA was hybridized on the 42K GeneChip set and the HuGeneFL array for twenty-four hours at 60 rpm in a 45° C. hybridization oven. The chips were washed and stained with Streptavidin Phycoerythrin (SAPE) (Molecular Probes) in Affymetrix fluidics stations. To amplify staining, SAPE solution was added twice with an anti-streptavidin biotinylated antibody (Vector Laboratories) staining step in between. Hybridization to the probe arrays was detected by fluorometric scanning (Hewlett Packard Gene Array Scanner). Data was analyzed using Affymetrix GeneChip version 3.0 and Expression Data Mining Tool (EDMT) software (version 1.0).

Differential expression of genes between the allergic hypersensitive and normal cell or tissue samples was determined using the Affymetrix GeneChip set by the following criteria: (1) For each gene, Affymetrix GeneChip average difference values were determined by standard Affymetrix EDMT software algorithms, which also made “Absent” (=not detected), “Present” (=detected) or “Marginal” (=not clearly Absent or Present) calls for each GeneChip element. (2) All negative values (=Absent) were raised to a floor of +20 (positive 20) so that fold change calculations could be made where values were not already greater than or equal to +20. (3) Median levels of expression were compared between the normal control group and the allergic hypersensitive disease group to obtain greater than or equal 3-fold up/down values; (4). The median value for the higher expressing group needed to be greater or equal to 200 average difference units in order to be considered for statistical significance. (5) Genes passing the criteria of #1-4 were analyzed for statistical significance using a two-tailed T test and deemed statistically significant if p<0.05.

MC1 (AA447527) exhibited no significant changes in expression levels in mature mast cells between normal (represented by resting samples) and stimulated state (represented by activated samples), demonstrated by the 1.2 fold change (down). However, this gene was expressed at much higher levels in mast cells with repeated stimulations (represented by an 8-week reactivation sample). There was a 3 fold difference in expression between the 8-week reactivation sample and the 6-week resting samples and 6-week activation samples.

MC1 also exhibited mast cell specific expression profiles. MC1 was called present in all 15 mast cell samples, and the Average Difference values, as calculated by GeneExpress™, ranged from 2017 to 17792 with a mean value of 5671. In a survey of expression levels in 31 other normal tissue types (349 samples), MC1 was only called present in 10 of them and the Average Difference Values for those 10 samples were all below 200, which is considered to be the marginal background noise value for Affy GeneChip™.

### Example 2

Cloning of a Full Length Human cDNA Corresponding to the Differentially Expressed mRNA species of SEQ ID NO: 1

The full length cDNA having SEQ ID NO: 1 was obtained by the solution hybridization method. Briefly, a gene-specific oligo was designed based on the sequence of the EST fragment identified in Example 1. The oligo was labeled with biotin and used to hybridize with 5 μg of single strand plasmid DNA (cDNA recombinants) from a human resting mast cell library following the procedures from the Gene Trapper kit obtained from Life Technologies. The hybridized cDNAs were separated by streptavidin-conjugated beads and eluted by TE buffer. The eluted cDNA was converted to double strand plasmid DNA and used to transform E. coli cells (DHα5). 94 clones were screened by PCR using gene specific primers designed from the EST sequence. 61 clones were positive for the sequence. After positive selection, the cDNA clone was subjected to DNA sequencing.

The nucleotide sequences of the full-length human cDNA corresponding to the differentially regulated mRNAs detected above is set forth in SEQ ID NO: 1. The cDNA comprises 3762 base pairs, with an open reading frame at nucleotides 25-432 encoding a protein of 135 amino acids (nucleotides 25-429 without the TAA stop codon). The amino acid sequence corresponding to the encoded protein is set forth in SEQ ID NO: 2.

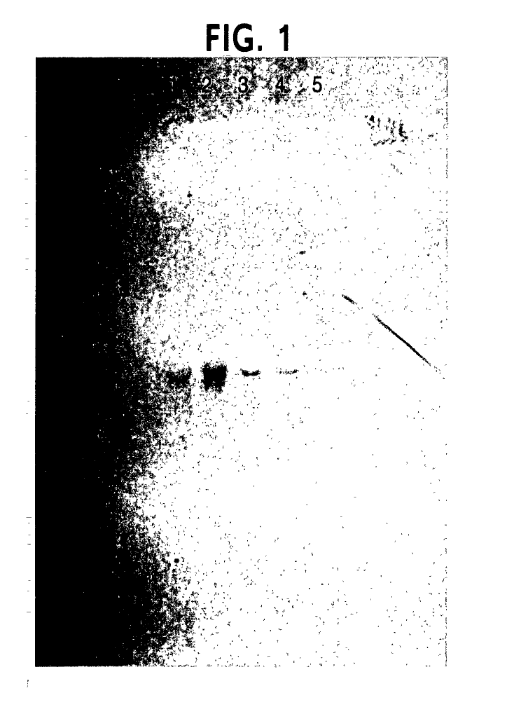

FIG. 1 shows relative AA447527 (MC1) mRNA levels determined via Northern Blot in various normal and leukemic cell cultures as described above. A probe based on SEQ ID NO: 1 (randomly labeled) was exposed to human mRNA blots for 1 hour at 68° C. in Clontech ExpressHyb hybridization buffer, following standard methodology as described by Sambrook et al. (1989). The highest expression level was observed in mature mast cell cultures, particularly the activated mast cell cultures, with lower but significant expression observed in leukemic mast cell, basophil and megakaryocyte cell cultures.

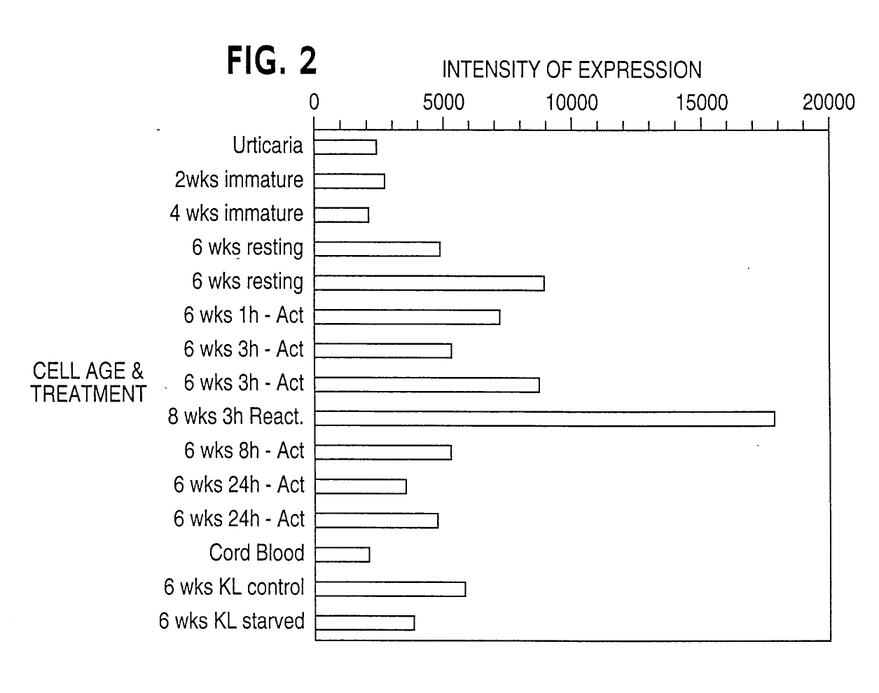

As shown in FIG. 2 the relative expression of AA447527 (MC1) was quantified using Quantitative RT-PCR in RNA samples that were from mast cells and cultured under conditions permissive to differentiation into mature mast cells at 6 weeks. Cells were sampled at various times during maturation and upon activation of the mature cells by presentation of an antigen. The antigen was added at time zero and the cells prepared for assay at 1, 3, 8 or 24 hours later. Experienced cells were prepared by the addition of IgE/Ag two times, once at six weeks, and again at seven weeks. The final stimulation at eight weeks was followed by collection after three hours of stimulation. For comparison, cell samples were prepared from a patient with urticaria, from cord blood and from cells grown in the absence of a required cytokine, kit ligand.

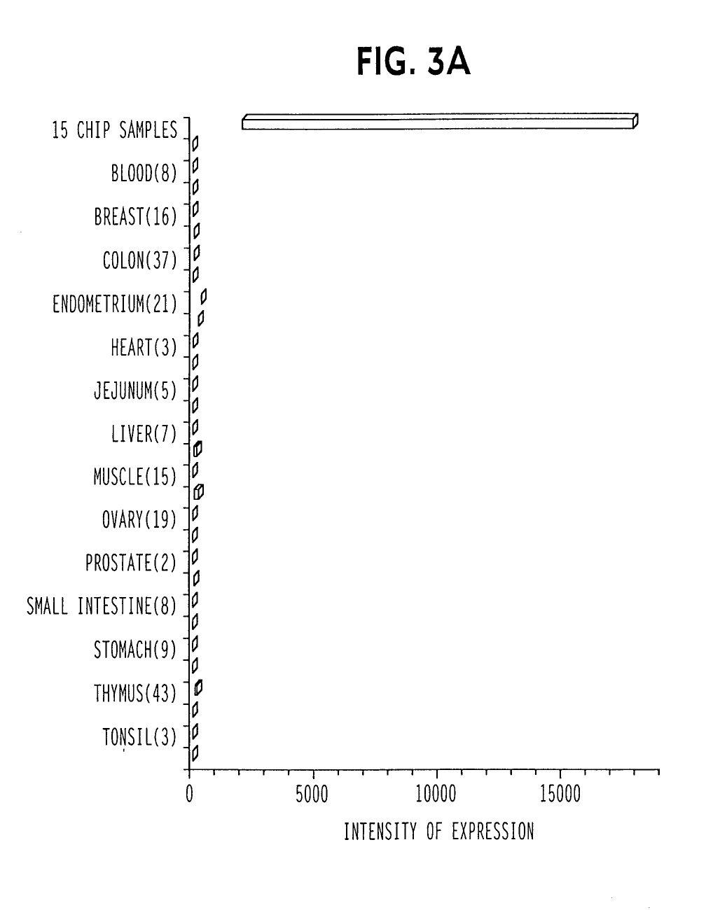

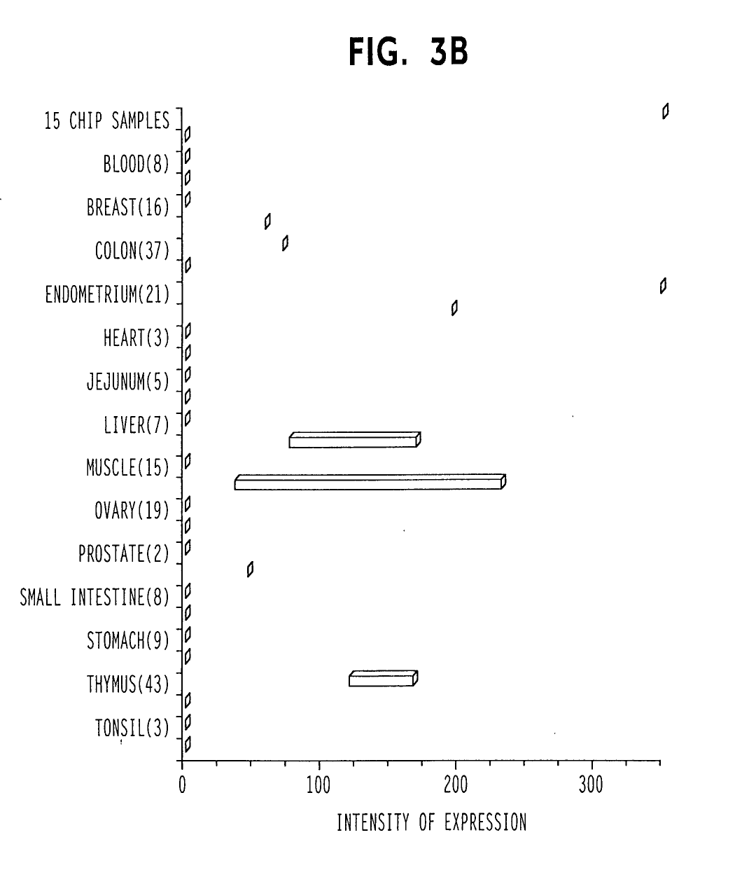

Expression was assessed across a panel of normal human tissue by (see FIG. 3A and FIG. 3B) using Affymetrix GeneChip™ 42K chip set. Different numbers of tissue samples were present in each group of tissues. The average difference values obtained from each sample were gathered and the median for each tissue group was plotted.

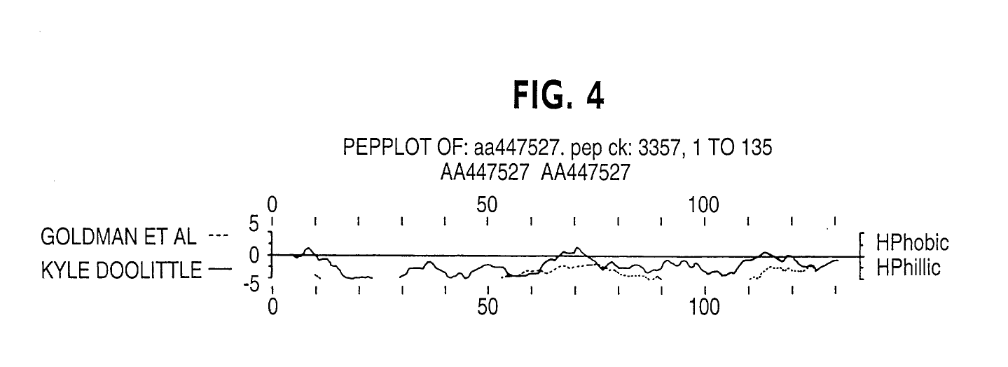

Hydrophobicity analysis (see FIG. 4) of the amino acid sequence suggest that the novel polypeptide of SEQ ID NO: 2 is largely hydrophilic. The protein consists of 21 basic amino acids, 23 acidic amino acids, 50 polar amino acids and 23 hydrophobic amino acids.

### Example 3

Identification of Differentially Expressed mRNA in Mast Cells Generated in vitro from Precursors

Differential expression of genes between activated and resting mast cell samples was determined using the Affymetrix GeneChip set by the following criteria: (1) For each gene, Affymetrix GeneChip average difference values were determined by standard Affymetrix EDMT software algorithms, which also made “Absent” (=not detected), “Present” (=detected) or “Marginal” (=not clearly Absent or Present) calls for each GeneChip element. (2) All negative values (=Absent) were raised to a floor of +20 (positive 20) so that fold change calculations could be made where values were not already greater than or equal to +20. (3) Median levels of expression were compared between the normal control group and the allergic hypersensitive disease group to obtain greater than or equal to 3-fold up/down values; (4). The median value for the higher expressing group needed to be greater or equal to 200 average difference units in order to be considered for statistical significance. (5) Genes passing the criteria of #1-4 were analyzed for statistical significance using a two-tailed T test and deemed statistically significant if p<0.05.

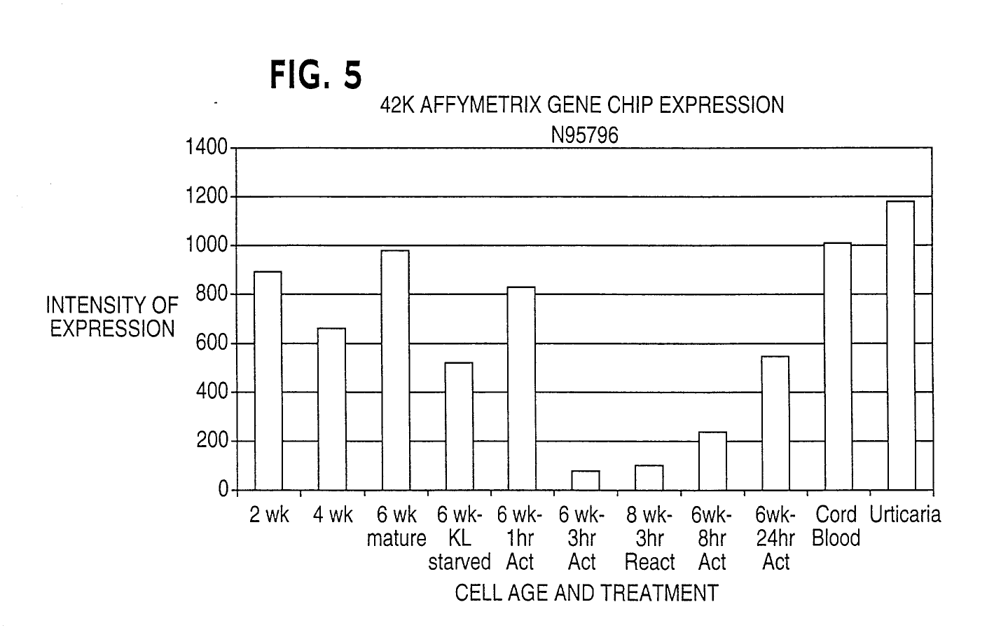

As shown in FIG. 5, MC14 (N95796) exhibited no significant changes in expression level at different stages of maturation in normal mast cells, nor was there a significant difference among normal, cord blood or urticaria derived mast cells. However, this gene was expressed at much lower levels in mast cells that had been activated for 3 or 8 hours or reactivated with IgE and antigen. There was approximately a 17-fold difference in expression level between the resting and the 3 hour activated samples. This pattern of expression, down regulation with activation, has been observed for other known mast cell signal transduction target genes on the Affymetrix GeneChip, and is therefore a useful criteria for selection of genes or ESTs for further characterization.

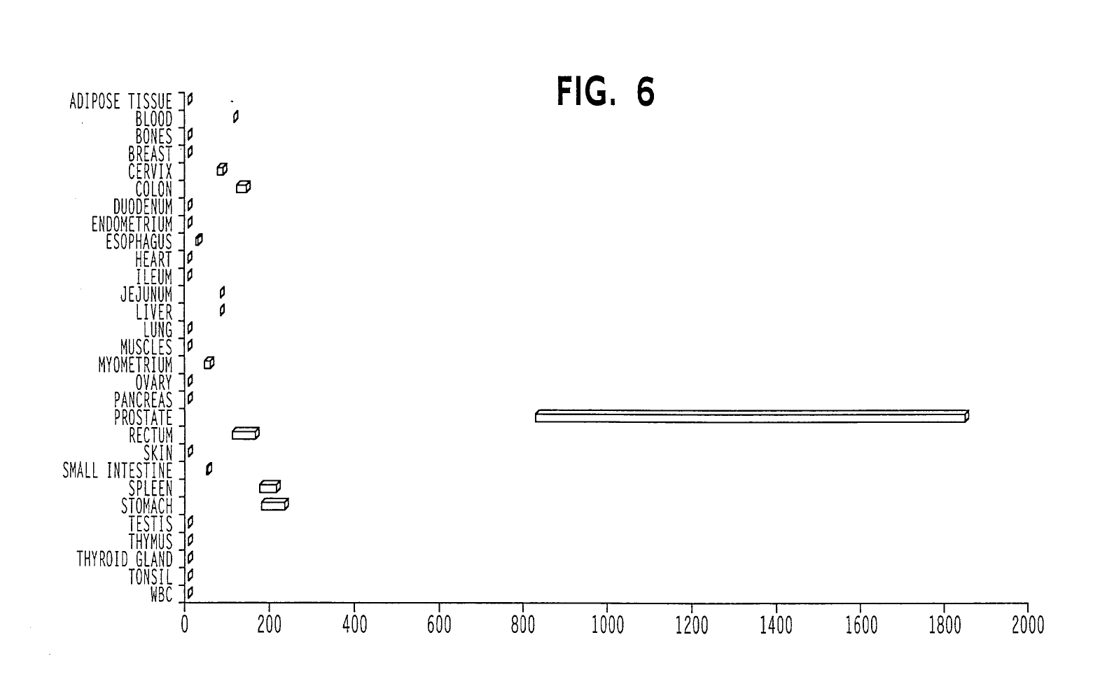

MC14 also exhibited tissue specific and mast cell specific expression profiles. Expression was assessed across a panel of normal human tissues and across a panel of mast cell samples (data not shown) by using Affymetrix 42K GeneChip™ set (see FIG. 6). Different numbers of tissue samples were present in each group of tissues. The average difference values obtained from each sample were gathered and calculated and the median for each tissue group was plotted using GeneExpress™. MC14 was called present in 14 of the 15 mast cell samples, and the calculated Average Difference values ranged from 82 to 1188 with a mean value of 643. The only absent call was in an activated sample. In a survey of expression levels in 29 other normal tissue types (427 samples), MC14 was only called present in 24 of them and the Average Difference Values for 22 of those 24 samples were all below 200, which is considered to be the marginal background noise value for Affy GeneChip™. The two samples with a significant level of expression were the 2 prostate samples with a mean expression level of 913. These results demonstrate the relative mast cell selective expression of MC14 in the mast cell lineage.

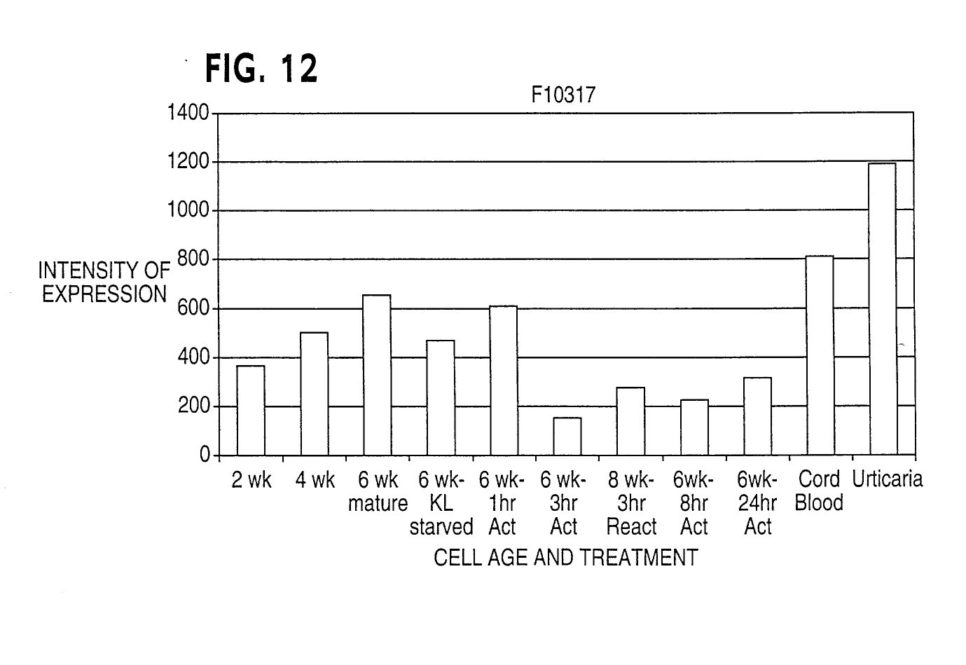

As shown in FIG. 12, in normal mast cells, another gene, referred to as MC16 (F10317) exhibited a gradually increasing level of expression with increasing levels of maturation. The expression level in urticaria derived mast cells was almost twice that of mature cultured mast cells. This gene was expressed at lower levels in mast cells that had been activated for 3 hours with IgE and antigen, although the level of expression gradually increased with time following activation (8 hour and 24 hour time point samples).

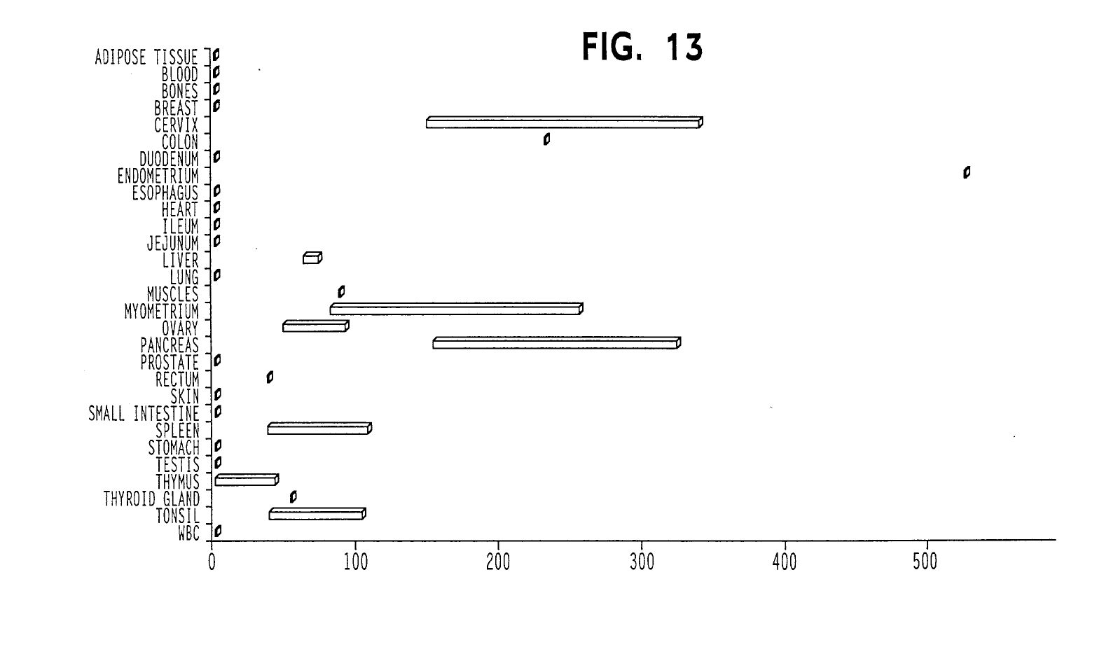

MC16 also exhibited tissue-specific and mast cell specific expression profiles. Expression was assessed across a panel of normal human tissues by using Affymetrix GeneChip™ 42K chip set (see FIG. 13). Different numbers of tissue samples were present in each group of tissues. The average difference values obtained from each sample were gathered and calculated and the median for each tissue group was plotted using GeneExpress™. MC16 was called present in all of the 15 mast cell samples (data not shown), and the calculated Average Difference values ranged from 162 to 1189. In a survey of expression levels in 29 other normal tissue types (434 samples total), MC16 was called present in 72 samples and was expressed at a significant level in cervix, endometrium, myometrium, pancreas, and colon.

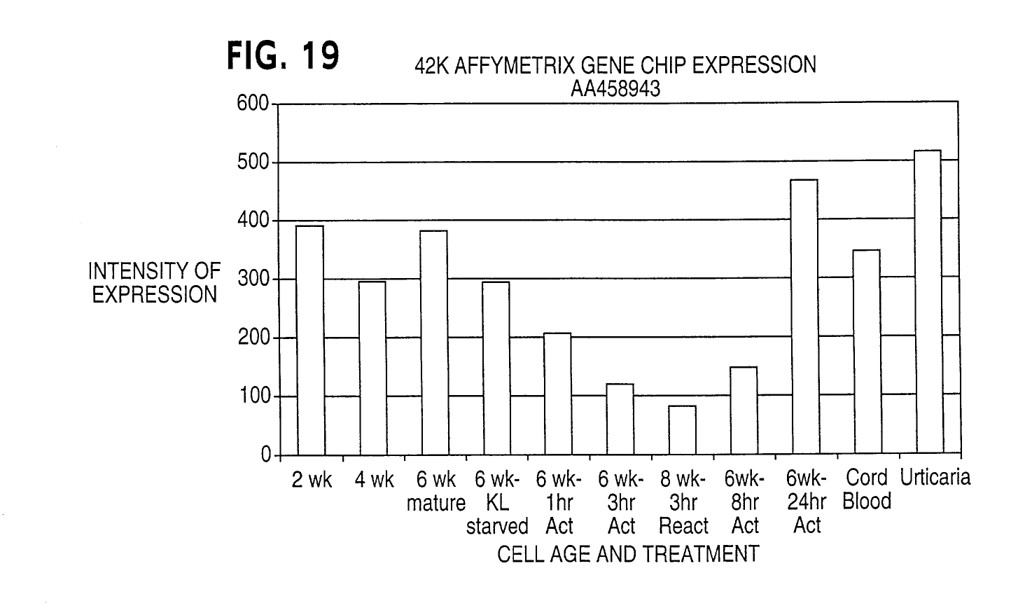

As shown in FIG. 19, a gene referred to as MC17 (AA458943) exhibited no significant changes in expression level at different stages of maturation in normal mast cells, nor was there a significant difference among normal, cord blood or urticaria derived mast cells. However, this gene was expressed at much lower levels in mast cells that had been activated for 3 or 8 hours or reactivated with IgE and antigen. There was approximately a 3-fold difference in expression level between the resting and the 3 hour activated samples.

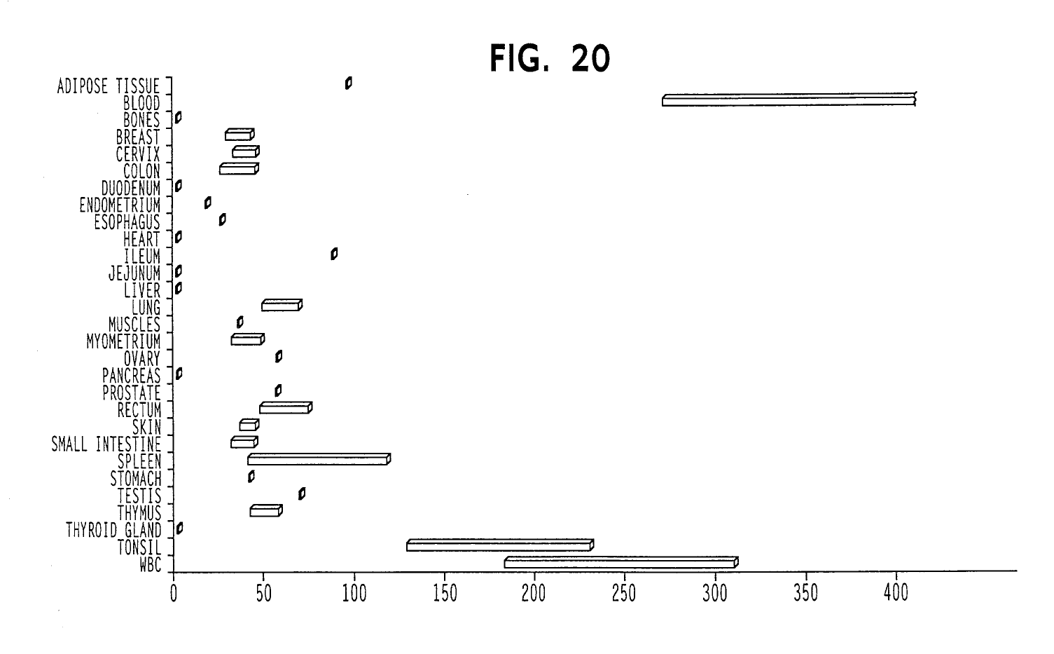

MC17 also exhibited tissue specific and mast cell specific expression profiles. Expression was assessed across a panel of normal human tissues and across a panel of mast cell samples (data not shown) by using Affymetrix 42K GeneChip™ set (see FIG. 20). Different numbers of tissue samples were present in each group of tissues. The average difference values obtained from each sample were gathered and calculated and the median for each tissue group was plotted using GeneExpress™. MC17 was called present in all of the 15 mast cell samples, and the calculated Average Difference values ranged from 88 to 523 with a mean value of 310. In a survey of expression levels in 29 other normal tissue types (437 samples), MC17 was called present in only 157 of them. MC17 was primarily expressed in hematopoietic cells/tissues, such as white blood cells, whole blood, spleen, and tonsil. The mean expression levels for these tissues ranged from 96 to 351. These results demonstrate that in addition to the high level of expression in mast cells, MC17 is also expressed in other hematopoietic cell types.

### Example 4

Cloning of Full Length Human cDNAs Corresponding to the Differentially Expressed mRNA Species

The full-length cDNA having SEQ ID NO: 3 was obtained by the solution hybridization method. Briefly, a gene-specific oligo was designed based on the sequence of the N95796 EST fragment identified in Example 3. The oligo was labeled with biotin and used to hybridize with 5 μg of single strand plasmid DNA (cDNA recombinants) from a human resting mast cell library following the procedures from the Gene Trapper kit obtained from Life Technologies. The hybridized cDNAs were separated by streptavidin-conjugated beads and eluted by TE buffer. The eluted cDNA was converted to double strand plasmid DNA and used to transform E. coli cells (DHα5). 95 clones were screened by PCR using gene specific primers designed from the EST sequence. 80 clones were positive for the sequence. Five clones were selected and the one with the longest insert was subjected to DNA sequencing.

The nucleotide sequences of the full-length human cDNA corresponding to the differentially regulated mRNAs detected above is set forth in SEQ ID NO: 3. The cDNA comprises 3663 base pairs, with a first open reading frame at nucleotides 294-743 encoding a protein of 150 amino acids (nucleotides 294-746 with the TAA stop codon). The amino acid sequence corresponding to the encoded protein is set forth in SEQ ID NO: 4. The cDNA has a second open reading frame at nucleotides 1238-2215 encoding a protein of 326 amino acids (nucleotides 1238-2218 with the TAG stop codon). The amino acid sequence corresponding to the second encoded protein is set forth in SEQ ID NO: 5.

Sequence homology searching (Blastn) revealed nucleotide similarity to the genomic sequence disclosed under GenBank Accession No. AL365261, this sequence displaying homology to the complementary strand of SEQ ID NO: 3 over 1999 nucleotides (nucleotides 16-2014). The disclosed sequence, however, does not identify the presence of any open reading frames or coding sequences.

Another sequence disclosed in GenBank, Accession No. AF109303, shows partial nucleotide sequence homology to the 5′ end of the first open reading frame of SEQ ID NO: 3. Accession No. AF109303 is an EST, annotated as a prostate cancer associated protein, and does not correspond to any open reading frames or coding sequences.

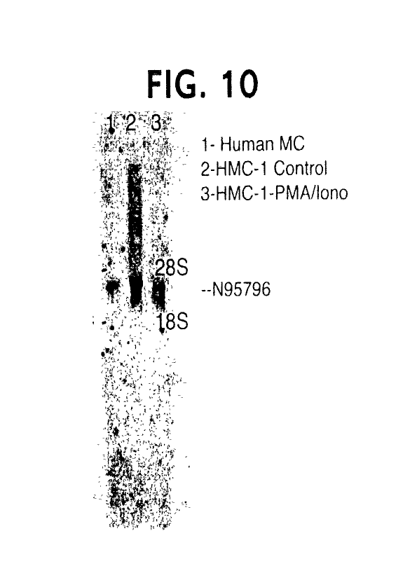

FIG. 10 shows relative N95796 mRNA levels determined via Northern Blot using total RNA from primary mast cells and a mast cell leukemia cell line, resting or activated with ionomycin and PMA. A probe based on SEQ ID NO: 3 (randomly labeled, using the Stratagene Prime-it II kit) was exposed to human mRNA blots (ClonTech mRNA blot-H) overnight at 42° C. in hybridization buffer, following standard methodology as described in Molecular Cloning a Laboratory Manual 2^{nd} edition, eds. Sambrook, Fritsch, and Maniatis, p 7.52. Expression of an approximately 3.6 kB transcript was observed in all three samples with the highest expression level observed in the resting leukemic mast cell line.

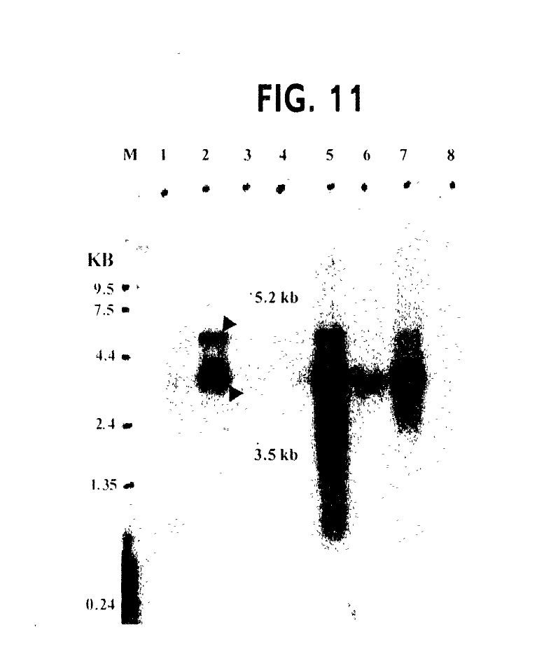

FIG. 11 is a Northern blot showing the detection of 2 mRNA species using isolated mRNA from various normal human tissues. Lane M) RNA markers; 1) heart; 2) brain; 3) placenta; 4) lung; 5) liver; 6) smooth muscle; 7) kidney; 8) pancreas. Using the more sensitize hybridization conditions (Church-Gilbert buffer, overnight at 65° C.) and mRNA in this tissue blot, a partially processed or alternatively spliced variant is also observed at 5.2 kB. Significant expression was observed in liver, brain, and kidney, with lower levels of expression in smooth muscle and lung.

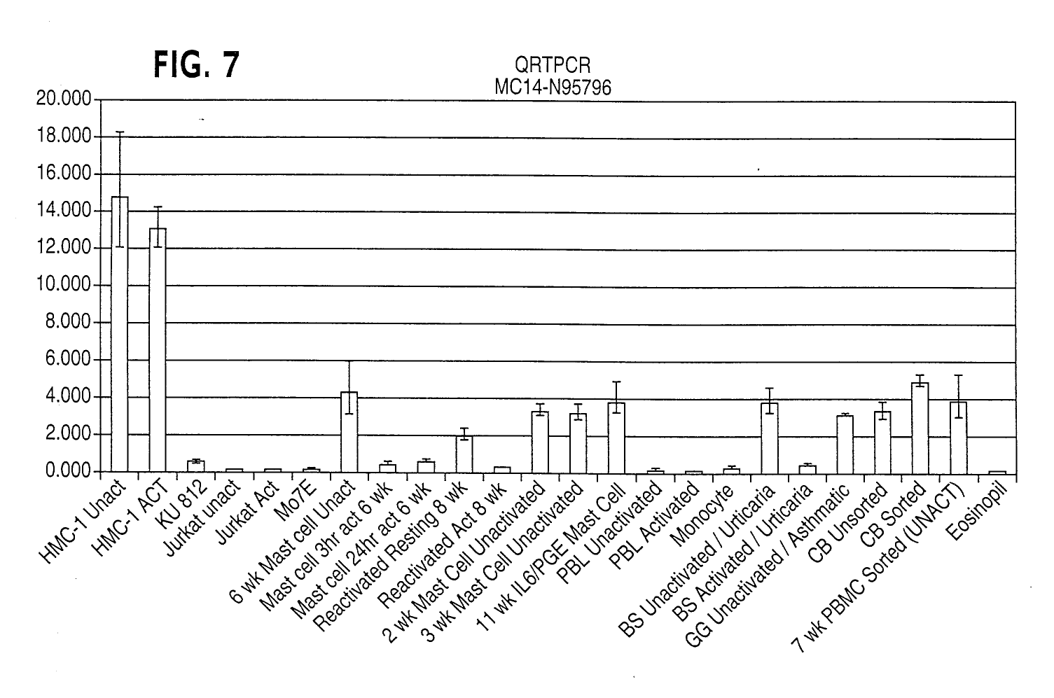

As shown in FIG. 7, the relative expression of N95796 was quantified using Quantitative RT-PCR on RNA samples from mast cells and mast cells cultured under conditions permissive to differentiation into mature mast cells at 6 weeks. The data is expressed as a ratio of the threshold cycle number for N95796 relative to dynamitin. Dynamitin is a control gene that has similar levels of expression over the samples in this set. Cells were sampled at various times during maturation and upon activation of the mature cells by presentation of an antigen. The antigen was added at time zero and the cells prepared for assay at 3 or 24 hours later. Multiply stimulated cells (reactivated) were prepared by the addition of IgE/Ag two times, once at six weeks, and again at seven weeks. The final stimulation at eight weeks was followed by collection after three hours of stimulation. For comparison, cell samples were prepared from a patient with urticaria, from cord blood and from cells grown in the absence of a required cytokine, kit ligand, for seven hours. These results confirmed the Affymetrix GeneChip data in a number of ways. For instance, expression of N95796 is down regulated with activation or reactivation in normal mast cells, as well as in urticaria donor derived cells. The data also confirmed the relative MC specificity of expression of N95796 in that it was detected at only a very low level in the non-mast cell samples in this set.

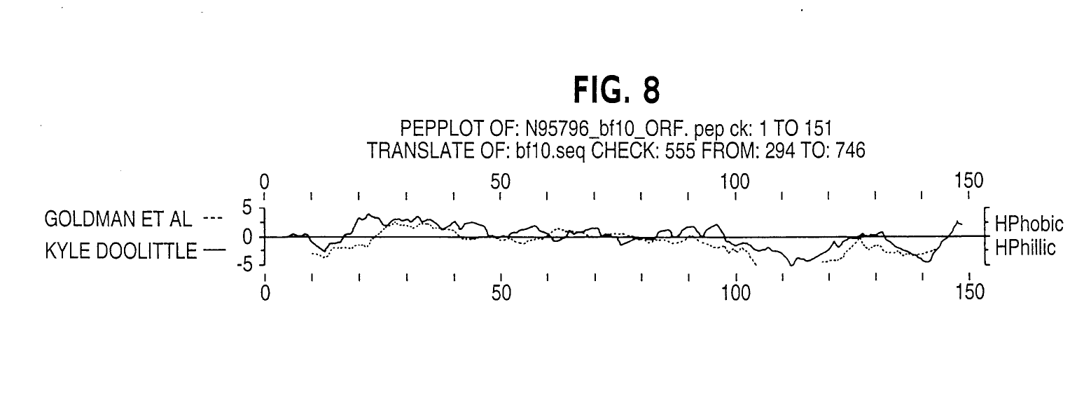

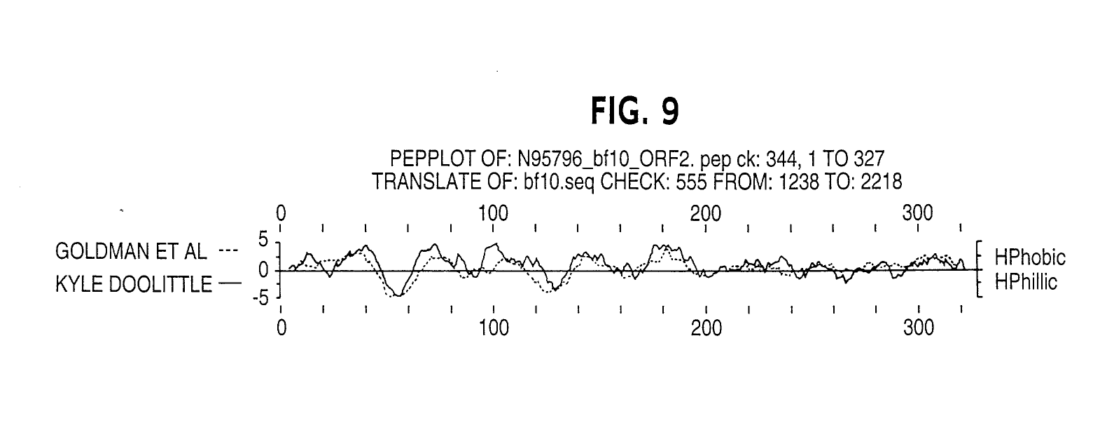

Hydrophobicity analysis (see FIGS. 8 and 9) of the amino acid sequence suggest that the novel polypeptide of SEQ ID NO: 4, 150 amino acids, is largely hydrophobic toward the amino terminus and largely hydrophilic toward the carboxy terminus. The novel polypeptide of SEQ ID NO: 5, however, 326 amino acids, contains alternating hydrophobic and hydrophilic regions in the amino-terminal half, while the carboxy-terminal half is largely neutral.

The full-length cDNA having SEQ ID NO: 6 was obtained by the solution hybridization method. Briefly, a gene-specific oligo was designed based on the sequence of the F10317 EST fragment identified in Example 3. The oligo was labeled with biotin and used to hybridize with 5 μg of single strand plasmid DNA (cDNA recombinants) from a human resting mast cell library following the procedures from the Gene Trapper kit obtained from Life Technologies. The hybridized cDNAs were separated by streptavidin-conjugated beads and eluted by TE buffer. The eluted cDNA was converted to double strand plasmid DNA and used to transform E. coli cells (DHα5). 95 clones were screened by PCR using gene specific primers designed from the EST sequence. 59 clones were positive for the sequence. Three clones with the longest inserts were selected for partial sequencing. The clone with the longest insert of 3.8 kb was completely sequenced.

The nucleotide sequences of the full-length human cDNA corresponding to the differentially regulated mRNAs detected above is set forth in SEQ ID NO: 6. The cDNA comprises 3743 base pairs, with an open reading frame at nucleotides 377-1951 encoding a protein of 524 amino acids (nucleotides 377-1948 without the TGA stop codon). The amino acid sequence corresponding to the encoded protein is set forth in SEQ ID NO: 2. Sequence homology searching (Blastn) revealed nucleotide similarity to the genomic sequence disclosed under GenBank Accession No. AK021475, this sequence displaying homology to SEQ ID NO: 6 over 2096 nucleotides (nucleotides 1622-3717). The disclosed sequence identifies an open reading frame corresponding to nucleotides 1740 to 2225 of SEQ ID NO: 6, although the disclosed open reading frame is out of frame with respect to SEQ ID NO: 6. No name or function is associated with the open reading frame in the disclosed sequence.

Another sequence disclosed in GenBank, Accession No. AC024008, shows nucleotide sequence homology in three regions of SEQ ID NO: 6, nucleotides 1450-1652, 1649-1896 and 1753-3730. The disclosed sequence does not correspond to any open reading frames or coding sequences.

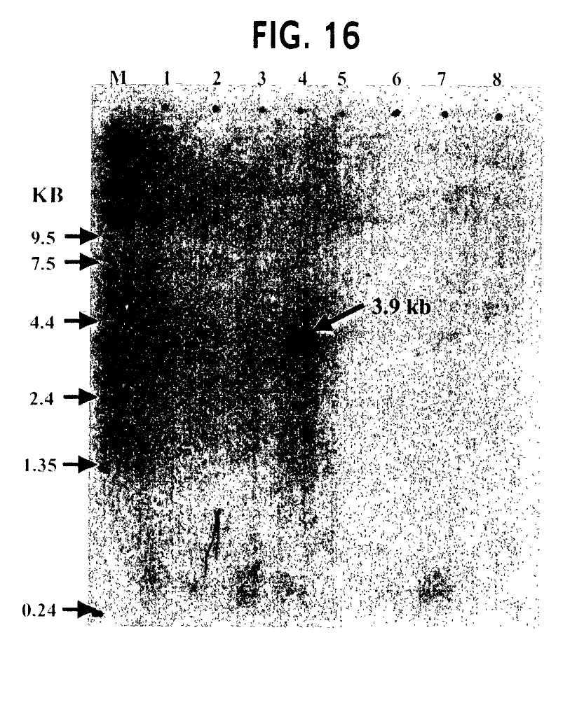

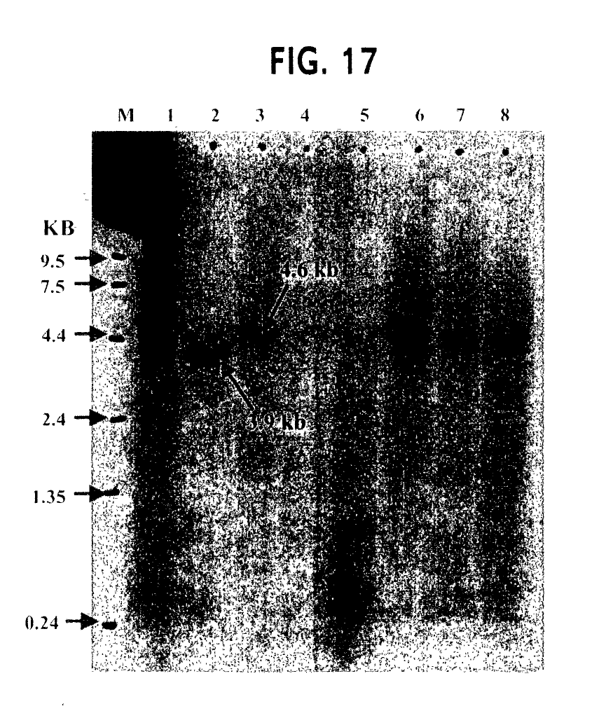

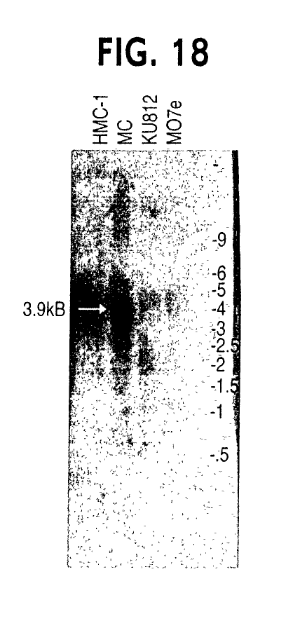

FIGS. 16, 17 and 18 are Northern blots showing the detection of 2 mRNA species using isolated mRNA from various normal human tissues, mast cells and cell lines. As mentioned above, FIG. 16 contains samples from spleen, thymus, prostate, testis, uterus, small intestine, colon and leukocytes, in lanes 1-8, respectively, while FIG. 17 contains samples from heart, brain, placenta, lung, liver, smooth muscle, kidney and pancreas, in lanes 1-8, respectively. FIG. 18 contains RNA from cultured human mast cells (MC) and the cell lines HMC-1, KU812, and MO7e. A probe based on SEQ ID NO: 6 (randomly labeled, using the Stratagene Prime-it II kit) was exposed to human mRNA blots (ClonTech mRNA, H4 Blot in FIG. 4, H Blot in FIG. 16) overnight at 65° C. in Church-Gilbert hybridization buffer, washing to final conditions of 0.1×SSC and 0.1% SDS. The MC and cell line blot in FIG. 18 was hybridized overnight at 42° C. in ULTRAhyb hybridization solution (Ambion), and washed to final conditions of 0.1×SSC and 0.1% SDS at 42° C. In FIG. 18 a major transcript of 3.9 kb is observed in MC. Expression of an approximately 3.9 kb transcript was observed in testicular and brain tissue, and, under these sensitive hybridization conditions, a partially processed or alternatively spliced variant of approximately 4.6 kb is observed in smooth muscle, kidney, pancreas and placenta as well as the KU812 and MO7e cell lines.

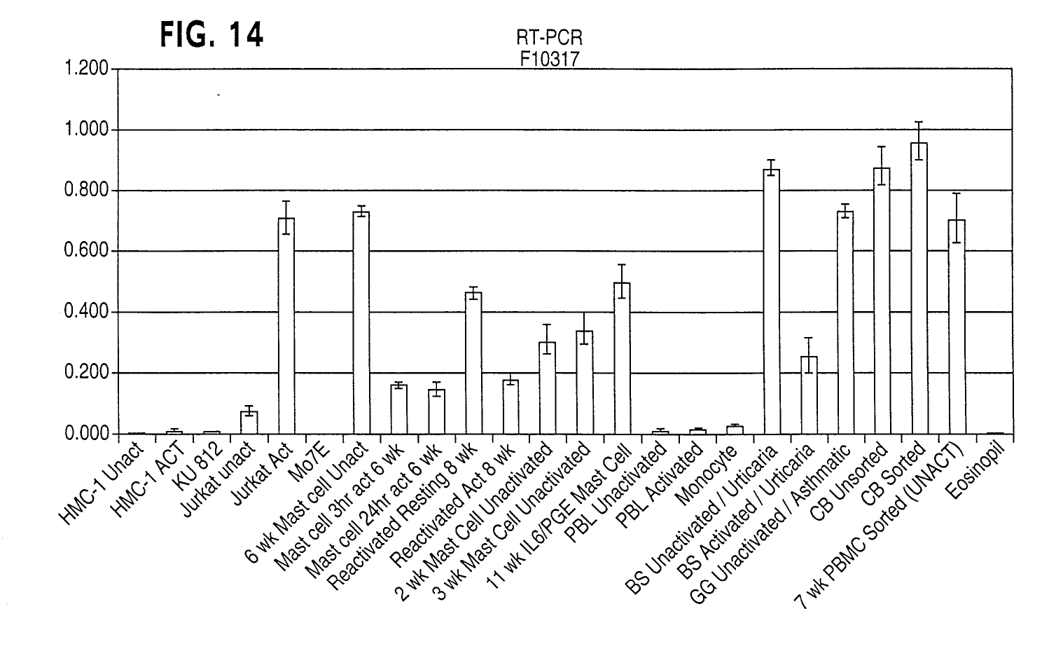

As shown in FIG. 14, the relative expression of F10317 was quantified using Quantitative RT-PCR on RNA samples from mast cells and mast cells cultured under conditions permissive to differentiation into mature mast cells at 6 weeks. The data is expressed as a ratio of the threshold cycle number for F10317 relative to dynamitin. Dynamitin is a control gene that has similar levels of expression over the samples in this set. Cells were sampled at various times during maturation and upon activation of the mature cells by presentation of an antigen. The antigen was added at time zero and the cells prepared for assay at 3 or 24 hours later. Multiply stimulated cells (reactivated) were prepared by the addition of IgE/Ag two times, once at six weeks, and again at seven weeks. The final stimulation at eight weeks was followed by collection after three hours of stimulation. For comparison, cell samples were prepared from a patient with urticaria, from cord blood and from cells grown in the absence of a required cytokine, kit ligand, for seven hours. These results confirmed the Affymetrix GeneChip data in a number of ways. This shows that expression of F10317 is down regulated with activation or reactivation in normal mast cells, although expression is up regulated in urticaria and asthmatic donor derived cells and increases gradually with age in cultured normal mast cells. The data also confirmed the relative MC specificity of expression of F10317. Only low levels of relative expression were seen in the non-mast cell hematopoietic cells samples in this set (KU812, Jurkat, MO7e, PBL, monocyte, eosinophil) except for the Jurkat cell line activated with PMA and ionomycin.

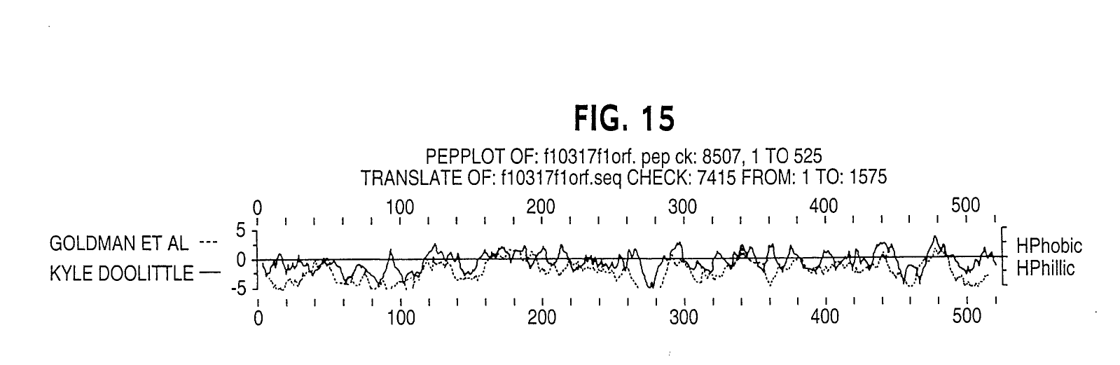

Hydrophobicity analysis (see FIG. 15) of the amino acid sequence suggests that the novel polypeptide of SEQ ID NO: 7, 524 amino acids, contains neither large hydrophobic nor large hydrophilic regions, although hydrophobic, neutral and hydrophilic residues are present throughout.

The full-length cDNA having SEQ ID NOS: 8 or 10 was obtained by the solution hybridization method. Briefly, a gene-specific oligo was designed based on the sequence of the AA458943 EST fragment identified in Example 3. The oligo was labeled with biotin and used to hybridize with 5 μg of single strand plasmid DNA (cDNA recombinants) from a human resting mast cell library following the procedures from the Gene Trapper kit obtained from Life Technologies. The hybridized cDNAs were separated by streptavidin-conjugated beads and eluted by TE buffer. The eluted cDNA was converted to double strand plasmid DNA and used to transform E. coli cells (DHα5).

The nucleotide sequences of the full-length human cDNA corresponding to the differentially regulated mRNAs detected above is set forth in SEQ ID NOS: 8 or 10. The cDNA comprises 1998 base pairs, with a first open reading frame at nucleotides 162-632 encoding a protein of 157 amino acids (nucleotides 162-635 with the TAG stop codon). The amino acid sequence corresponding to this encoded protein is set forth in SEQ ID NO: 9. The cDNA has a second open reading frame at nucleotides 373-648 encoding a protein of 92 amino acids (nucleotides 373-651 with the TGA stop codon). The amino acid sequence corresponding to the second encoded protein is set forth in SEQ ID NO: 11.

Sequence homology searching (Blastn) revealed nucleotide similarity to the genomic sequence disclosed under GenBank Accession No. AC025179, this sequence displaying homology to SEQ ID NOS: 8 or 10 over 1993 nucleotides (nucleotides 1-1993 of SEQ ID NOS: 8 or 10 show homology to nucleotides 91,476-93,468 of GenBank Accession No. AC025179). The disclosed sequence, however, is merely a section of six unordered pieces entered into GenBank and does not identify the presence of any open reading frames or coding sequences.

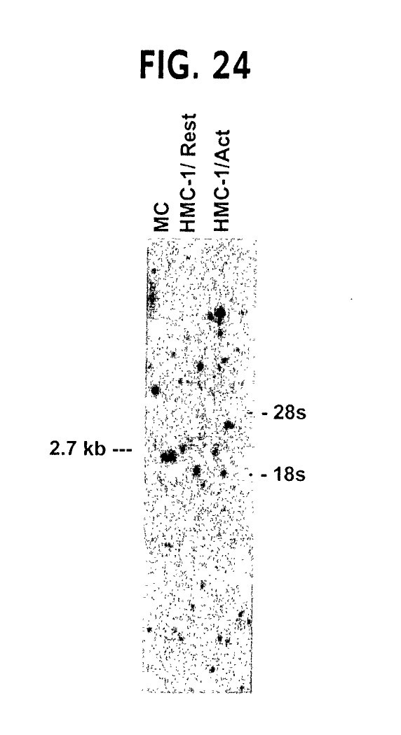

FIG. 24 shows relative AA458943 (MC17) mRNA levels determined via Northern Blot using total RNA from primary mast cells and a mast cell leukemia cell line, resting or activated with ionomycin and PMA. A probe based on SEQ ID NOS: 8 or 10 (randomly labeled, using the Stratagene Prime-it II kit) was exposed to human mRNA

blots overnight at 42° C. in hybridization buffer, following standard methodology as described in Molecular Cloning a Laboratory Manual 2^{nd} edition, eds. Sambrook, Fritsch, and Maniatis, p 7.52. Expression of an approximately 2.7 kb transcript was observed in mast cell RNA.

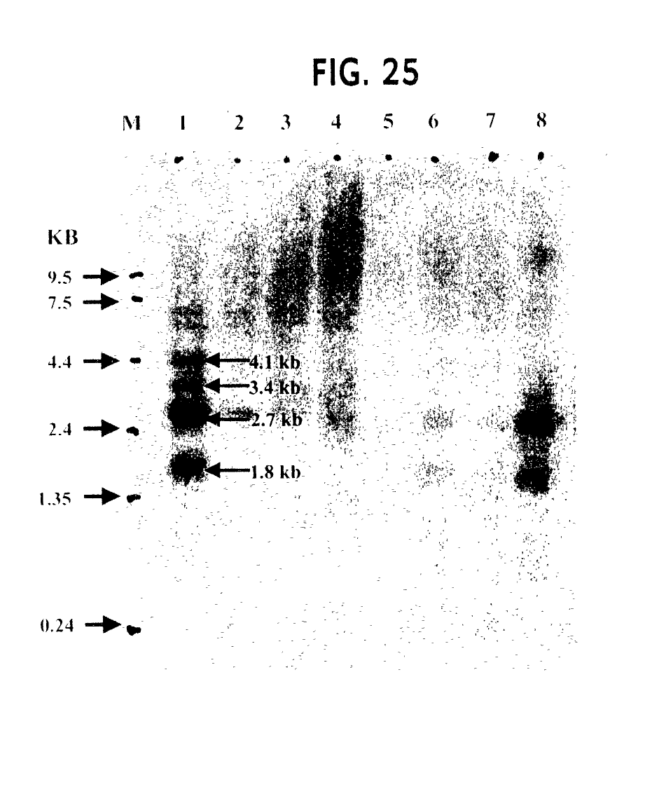

FIG. 25 is a Northern blot showing the detection of a major transcript of 2.7 kb with minor transcripts of 1.8 kb, 3.4 kb and 4.1 kb, using isolated mRNA from various normal human tissues (ClonTech). Lane M) RNA size markers; 1) spleen; 2) thymus; 3) prostate; 4) testis; 5) uterus; 6) small intestine; 7) colon; 8) leukocyte. Using the more sensitive hybridization conditions (Church-Gilbert buffer, overnight at 65° C.) and mRNA in this tissue blot, partially processed or alternatively spliced variants are observed. Significant expression was observed in spleen and in leukocytes, with lower levels of expression in small intestine.

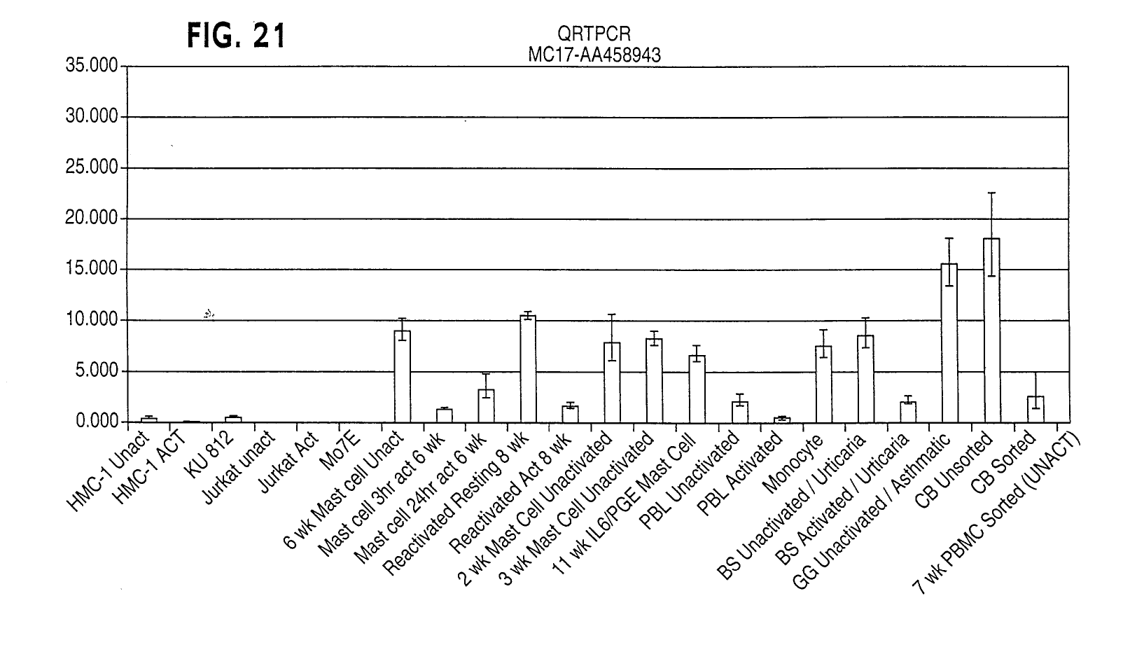

As shown in FIG. 21, the relative expression of AA458943 (MC17) was quantified using Quantitative RT-PCR on RNA samples from mast cells and mast cells cultured under conditions permissive to differentiation into mature mast cells at 6 weeks. The data is expressed as a ratio of the threshold cycle number for AA458943 relative to dynamitin. Dynamitin is a control gene that has similar levels of expression over the samples in this set. Cells were sampled at various times during maturation and upon activation of the mature cells by presentation of an antigen. The antigen was added at time zero and the cells prepared for assay at 3 or 24 hours later. Multiply stimulated cells (reactivated) were prepared by the addition of IgE/Ag two times, once at six weeks, and again at seven weeks. The final stimulation at eight weeks was followed by collection after three hours of stimulation. For comparison, cell samples were prepared from a patient with urticaria, from cord blood and from cells grown in the absence of a required cytokine, kit ligand, for seven hours. These results confirmed the Affymetrix GeneChip data in a number of ways. For instance, expression of AA458943 is down regulated with activation or reactivation in normal mast cells, as well as in urticaria donor derived cells. The data also confirmed the relative mast cell specificity of expression of AA458943 with expression in certain other hematopoietic cell types such as monocytes.

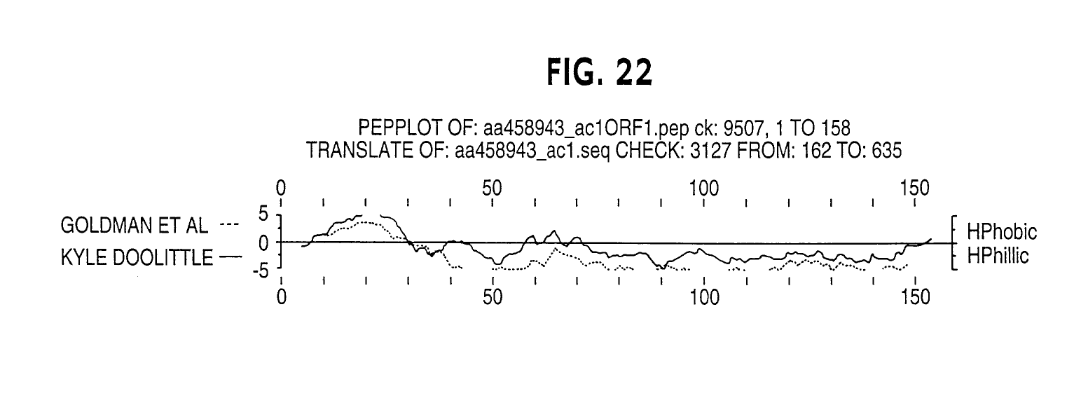

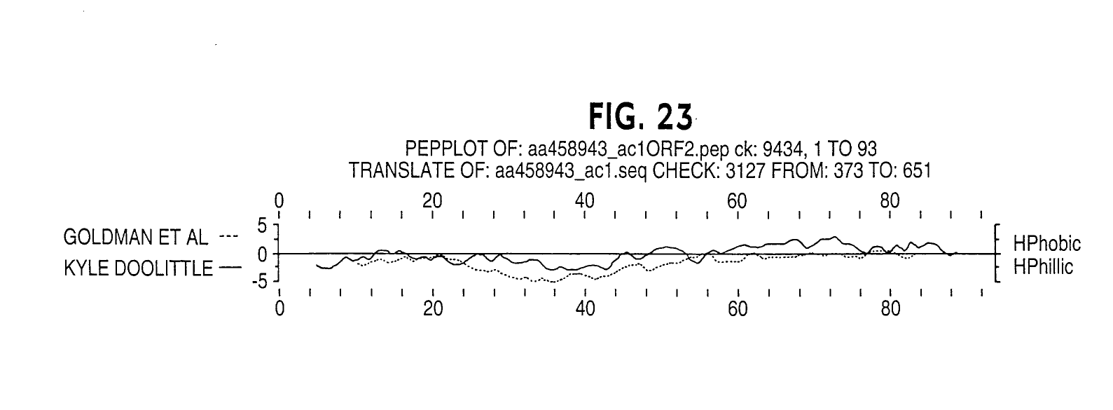

Hydrophobicity analysis (see FIG. 22) of the amino acid sequence encoded by the first open reading frame suggest that the novel polypeptide of SEQ ID NO: 9, 157 amino acids, contains a hydrophobic region toward the amino terminus, although the remainder of the polypeptide is more hydrophilic than hydrophobic. The novel polypeptide of SEQ ID NO: 11, 92 amino acids, (see FIG. 23) contains a hydrophilic regions near the amino terminus with a more hydrophobic region in the carboxy terminal third of the polypeptide.

### Example 5

### Expression of MC1 in Mast Cells

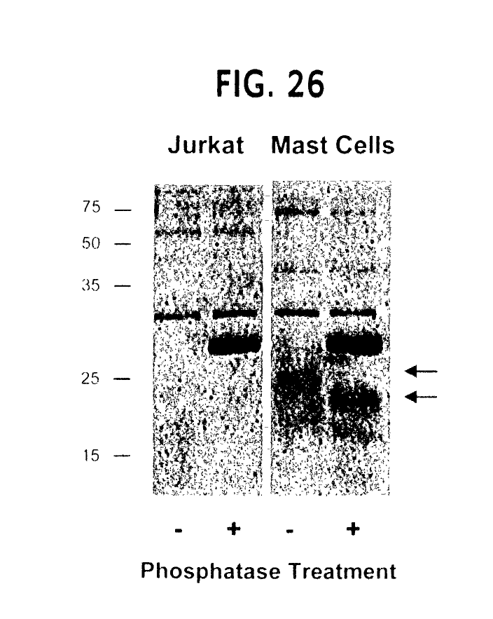

Mast cell specific expression of MC1 was demonstrated by a comparative western blot of primary cultured mast cells grown from peripheral blood versus Jurkat T cells as a negative control. Cell lysates of each cell type were prepared according to methods conventional in the art, separated by SDS-PAGE and the proteins were transferred onto membranes (FIG. 26). Aliquots from each cell type were treated with λ protein phosphatase prior to SDS-PAGE and transfer. Blots were probed with antisera raised to E. coli expressed MC1 protein. Mast cell lysates show the expression of an anti-MC1 reactive species not seen in the Jurkat control cells (top arrow). Phosphatase treatment of mast cell lysates exposes a faster migrating immunoreactive band (bottom arrow) which replaces the band seen in non-treated mast cells, demonstrating the presence of an anti-MC1 specific species in mast cells that is phosphorylated.

In order to assess the effect of MC1 overexpression in mast cells, primary cultured mast cells were infected with an adenoviral expression constructs containing the open reading frame of the MC1 cDNA (SEQ ID NO: 1). Jurkat T cells and CHO cells were identically infected as non-mast cell controls. Two days after infection cells were analyzed for viability by detecting the expression of the activation/apoptosis marker annexin V, which binds surface exposed phosphatidyl serine, and were further analyzed for mediator release.

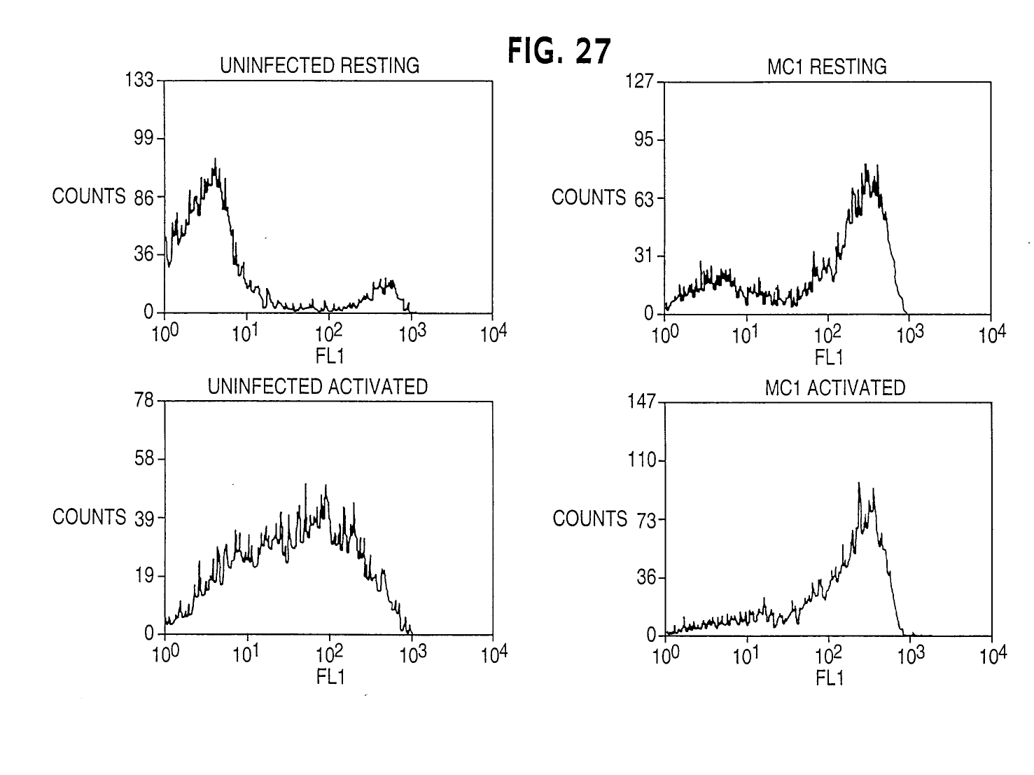

After two days of infection at a multiplicity of infection of 2500 the viability of primary mast cells infected with the MC1 vector was compromised relative to uninfected, or control mast cells infected with green fluorescent protein (GFP). Staining of mast cells for annexin V demonstrates the expression of the activation/apoptosis marker on the surface of mast cells overexpressing MC1 prior to activation, in contrast to uninfected cells (FIG. 27). Following activation, labeling with annexin V is observed on uninfected cells as expected, however the labeling of annexin V on MC1 infected cells is unchanged following activation compared to that seen on cells prior to activation. This effect of MC1 on viability and function appears to be selective for mast cells because no effect on viability or annexin V labeling was seen after infection of Jurkat and CHO control cells (not shown).

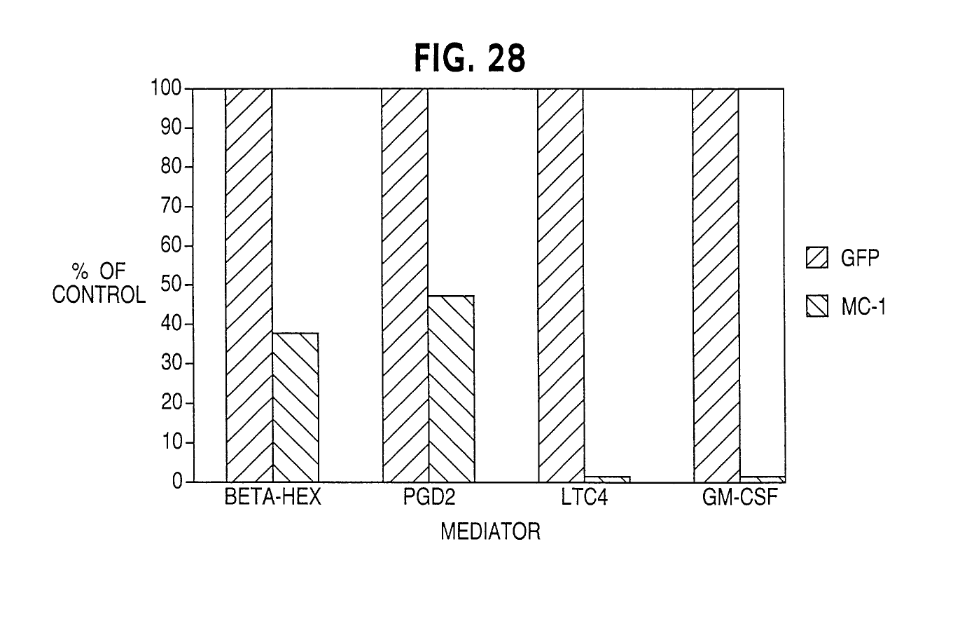

Further, mediator release following activation with IgE and antigen is significantly compromised following MC1 overexpression. FIG. 28 demonstrates that the release of the degranulation marker β hexoseaminidase is reduced compared to GFP infected or uninfected controls (not shown). The release of the lipid mediators LTC4 and PGD2 is also compromised relative to GFP infected controls as well as the synthesis of GM-CSF which is measured 18 hours after activation.

### Example 6

### MC14 Splice Variant Identification

An alternative splice variant was found to exist for the MC14 protein and gene. The open reading frame of the variant, MC14_altsplice2, occurs through the use of alternate splice sites within the introns of the genomic DNA sequence. This results in a variant form of the protein as depicted in SEQ ID NO: 13. The open reading frame encoding the protein (SEQ ID NO: 12) corresponds to a sequence previously been detected in prostate tissue (AY033593), but its expression profile in mast cells is predicted to be similar to that of MC14. The cDNA encoding MC14_altsplice2 predicts a multi-transmembrane protein with distant homology to a family of sucrose transporters in plants. The open reading frame disclosed herein as SEQ ID NO: 12 extends from nucleic acid residue 1 to residue 1662 (1659 without the TAG stop codon) of the sequence, encoding a protein of 553 amino acid residues (SEQ ID NO: 13). The open reading frame encoding SEQ ID NO: 13 shares the same start codon as the first open reading frame of SEQ ID NO: 3, which encodes SEQ ID NO: 4. SEQ ID NO: 13 is identical to SEQ ID NO: 4 through the glycine residue at position number 58 but diverges thereafter.

### Example 7

Detection of Allergic Hypersensitivity in a Patient

The expression level of a nucleic acid or protein of the invention is determined in a sample from a patient suspected of hypersensitivity before and/or after exposure to a potential antigen. The sample may be from an epithelial tissue such as a skin, respiratory tract or lung cell samples, or in urine or in blood samples. Epithelial tissue samples from a patient known to have allergic hypersensitivity and from normal subjects are used as positive and negative controls, respectively. A change in the level of expression of the nucleic acid or protein of the invention compared to the expression level in normal tissue may be indicative of allergic hypersensitivity.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

What is claimed is:

1. An isolated nucleic acid molecule selected from the group consisting of: (a) an isolated nucleic acid molecule that encodes the amino acid sequence of SEQ ID NO: 2, 4, 5, 7, 9, 11 or 13; (b) an isolated nucleic acid molecule that encodes a fragment of at least 6 amino acids of SEQ ID NO: 2, 4, 5, 7, 9, 11 or 13; (c) an isolated nucleic acid molecule which hybridizes to the complement of a nucleic acid molecule comprising SEQ ID NO: 1, 3, 6, 8, 10 or 12; (d) an isolated nucleic acid molecule which hybridizes to the complement of a nucleic acid molecule that encodes the amino acid sequence of SEQ ID NO: 2, 4, 5, 7, 9, 11 or 13; and (e) an isolated nucleic acid molecule that encodes a protein that exhibits at least about 35% amino acid sequence identity to SEQ ID NO: 2, 4, 5, 7, 9, 11 or 13.

2. An isolated nucleic acid molecule selected from the group consisting of: (a) an isolated nucleic acid molecule that encodes the amino acid sequence of SEQ ID NO: 2, 4, 5, 7, 9, 11 or 13; and (b) an isolated nucleic acid molecule that encodes the polypeptide of SEQ ID NO: 2, 4, 5, 7, 9, 11 or 13.

3. The isolated nucleic acid molecule of claim 1, wherein the nucleic acid molecule comprises nucleotides 25-429 of SEQ ID NO: 1.

4. The isolated nucleic acid molecule of claim 1, wherein the nucleic acid molecule consists of nucleotides 25-429 of SEQ ID NO: 1.

5. The isolated nucleic acid molecule of claim 1, wherein the nucleic acid molecule comprises nucleotides 25-432 of SEQ ID NO: 1.

6. The isolated nucleic acid molecule of claim 1, wherein the nucleic acid molecule comprises nucleotides 294-743 of SEQ ID NO: 3.

7. The isolated nucleic acid molecule of claim 1, wherein the nucleic acid molecule consists of nucleotides 294-743 of SEQ ID NO: 3.

8. The isolated nucleic acid molecule of claim 1, wherein the nucleic acid molecule comprises nucleotides 294-746 of SEQ ID NO: 3.

9. The isolated nucleic acid molecule of claim 1, wherein the nucleic acid molecule comprises nucleotides 1238-2215 of SEQ ID NO: 3.

10. The isolated nucleic acid molecule of claim 1, wherein the nucleic acid molecule consists of nucleotides 1238-2215 of SEQ ID NO: 3.

11. The isolated nucleic acid molecule of claim 1, wherein the nucleic acid molecule comprises nucleotides 1238-2218 of SEQ ID NO: 3.

12. The isolated nucleic acid molecule of claim 1, wherein the nucleic acid molecule comprises nucleotides 377-1948 of SEQ ID NO: 6.

13. The isolated nucleic acid molecule of claim 1, wherein the nucleic acid molecule consists of nucleotides 377-1948 of SEQ ID NO: 6.

14. The isolated nucleic acid molecule of claim 1, wherein the nucleic acid molecule comprises nucleotides 377-1951 of SEQ ID NO: 6.

15. The isolated nucleic acid molecule of claim 1, wherein the nucleic acid molecule comprises nucleotides 162-632 of SEQ ID NO: 8.

16. The isolated nucleic acid molecule of claim 1, wherein the nucleic acid molecule consists of nucleotides 162-632 of SEQ ID NO: 8.

17. The isolated nucleic acid molecule of claim 1, wherein the nucleic acid molecule comprises nucleotides 162-635 of SEQ ID NO: 8.

18. The isolated nucleic acid molecule of claim 1, wherein the nucleic acid molecule comprises nucleotides 373-648 of SEQ ID NO: 10.

19. The isolated nucleic acid molecule of claim 1, wherein the nucleic acid molecule consists of nucleotides 373-648 of SEQ ID NO: 10.

20. The isolated nucleic acid molecule of claim 1, wherein the nucleic acid molecule comprises nucleotides 373-651 of SEQ ID NO: 10.

21. The isolated nucleic acid molecule of claim 1, wherein the nucleic acid molecule consists of nucleotides 1-1662 of SEQ ID NO: 12.

22. The isolated nucleic acid molecule of claim 1, wherein the nucleic acid molecule comprises nucleotides 1-1662 of SEQ ID NO: 12.

23. The isolated nucleic acid molecule of any one of claims 1-22, wherein said nucleic acid molecule is operably linked to one or more expression control elements.

24. A vector comprising an isolated nucleic acid molecule of any one of claims 1-22.

25. A host cell transformed to contain the nucleic acid molecule of any one of claims 1-22.

26. A host cell comprising the vector of claim 24.

27. The host cell of claim 26, wherein said host is selected from the group consisting of prokaryotic host cells and eukaryotic host cells.

28. A method for producing a polypeptide comprising culturing a host cell transformed with the nucleic acid molecule of any one of claims 1-22 under conditions in which the protein encoded by said nucleic acid molecule is expressed.

29. The method of claim 28, wherein said host cell is selected from the group consisting of prokaryotic host cells and eukaryotic host cells.

30. An isolated polypeptide produced by the method of claim 28.

31. An isolated polypeptide or protein selected from the group consisting of an isolated polypeptide comprising the amino acid sequence of SEQ ID NO: 2, 4, 5, 7, 9, 11 or 13, an isolated polypeptide comprising a fragment of at least 6 amino acids of SEQ ID NO: 2, 4, 5, 7, 9, 11 or 13, an isolated polypeptide comprising conservative amino acid substitutions of SEQ ID NO: 2, 4, 5, 7, 9, 11 or 13 or naturally occurring amino acid sequence variants of SEQ ID NO: 2, 4, 5, 7, 9, 11 or 13, and an isolated polypeptide exhibiting at least about 35% amino acid sequence identity with SEQ ID NO: 2, 4, 5, 7, 9, 11 or 13.

32. An isolated polypeptide or protein selected from the group consisting of an isolated polypeptide comprising the amino acid sequence of SEQ ID NO: 2, 4, 5, 7, 9, 11 and 13.

33. An isolated antibody that binds to a polypeptide of claim 30, 31 or 32.

34. An antibody of claim 33 wherein said antibody is a monoclonal or a polyclonal antibody.

35. A method of identifying an agent which modulates the expression of a nucleic acid encoding a protein of claim 31 or 32, comprising:
exposing cells which express the nucleic acid to the agent; and
determining whether the agent modulates expression of said nucleic acid, thereby identifying an agent which modulates the expression of a nucleic acid encoding the protein.

36. A method of identifying an agent which modulates at least one activity of a protein of claim 31 or 32, comprising:
exposing cells which express the protein to the agent;
determining whether the agent modulates at least one activity of said protein, thereby identifying an agent which modulates at least one activity of the protein.

37. The method of claim 36, wherein the agent modulates one activity of the protein.

38. A method of identifying binding partners for a protein of claim 31 or 32, comprising:
exposing said protein to a potential binding partner; and
determining if the potential binding partner binds to said protein, thereby identifying binding partners for the protein.

39. A method of modulating the expression of a nucleic acid encoding a protein of claim 31 or 32, comprising:
administering an effective amount of an agent which modulates the expression of a nucleic acid encoding the protein.

40. A method of modulating at least one activity of a protein of claim 31 or 32, comprising:
administering an effective amount of an agent which modulates at least one activity of the protein.

41. A non-human transgenic animal modified to contain the nucleic acid molecule of any of claims 1-22.

42. The transgenic animal of claim 41, wherein the nucleic acid molecule contains a mutation that prevents expression of the encoded protein.

43. A method of diagnosing a disease state in a subject, comprising determining the level of expression of a nucleic acid molecule or protein of any one of claims 1-22, 31 or 32.

44. The method of claim 43, wherein the disease state is allergic hypersensitivity.

45. The method of claim 43, wherein the disease state is seasonal rhinitis

46. The method of claim 43, wherein the disease state is asthma.

47. The method of claim 43, wherein the disease state is urticaria or atopic dermatitis.

48. The method of claim 43, wherein the disease state is mastocytosis.

49. A composition comprising an isolated nucleic acid molecule of claim 1 or 2 and an aqueous carrier.

50. A method for the treatment or prevention of a disease state in a subject, comprising administering to said subject an effective amount of a nucleic acid molecule or protein of any one of claims 1-22, 31 or 32 or an agonist or antagonist thereof, thereby effecting said treatment or prevention of a disease state in said subject.

51. The method of claim 50, wherein the disease state is allergic hypersensitivity.

52. The method of claim 50, wherein the disease state is seasonal rhinitis.

53. The method of claim 50, wherein the disease state is asthma.

54. The method of claim 50, wherein the disease state is urticaria or atopic dermatitis.

55. The method of claim 50, wherein the disease state is mastocytosis.

56. A computer system comprising
(a) a database containing information identifying the expression level in a tissue or at least one mast cell of a set of nucleic acids comprising at least one nucleic aid sequence selected from the group consisting of SEQ ID NOs: 1, 3, 6, 8, 10 and 12 or a complement thereof, and
(b) a user interface to view the information.

57. A computer system comprising:
(a) a database containing information identifying the expression level in a tissue or at least one mast cell of a set of nucleic acids comprising at least one nucleic aid sequence encoding a protein selected from the group consisting of SEQ ID NOs: 2, 4, 5, 7, 9, 11 and 13 or a complement of said nucleic acid sequence; and
(b) a user interface to view the information.

58. The computer system of claim 56 or 57, wherein the database further comprises sequence information for said at least one nucleic acid sequence.

59. The computer system of claim 56 or 57, wherein the database further comprises information identifying the expression level for said at least one nucleic acid sequence in at least one normal mast cell.

60. A computer system of claim 56 or 57, wherein the database further comprises information identifying the expression level of said at least one nucleic acid sequence in at least one mast cell from a patient with allergic hypersensitivity.

61. A computer system of claim 60, wherein the patient with allergic hypersensitivity has rhinitis, asthma or urticaria.

62. A computer system of claim 56 or 57, further comprising records including descriptive information from an external database, which information correlates said genes to records in the external database.

63. A computer system of claim 62, wherein the external database is GenBank.

64. A method of using a computer system of claim 56 to present information identifying the expression level in a tissue or at least one mast cell of a set of nucleic acids comprising at least one nucleic aid sequence selected from the group consisting of SEQ ID NOs: 1, 3, 6, 8, 10 and 12 or a complement thereof, comprising comparing the expression level of at least one nucleic aid sequence selected from the group consisting of SEQ ID NOs: 1, 3, 6, 8, 10 and 12 or a complement thereof in the tissue or at least one mast cell to the level of expression of the nucleic acid sequence in the database.

65. A method of using a computer system of claim 57 to present information identifying the expression level in a tissue or at least one mast cell of a set of nucleic acids comprising at least one nucleic aid sequence encoding a protein selected from the group consisting of SEQ ID NOs: 2, 4, 5, 7, 9, 11 and 13 or a complement of said nucleic acid sequence, comprising comparing the expression level of at least one nucleic aid sequence encoding a protein selected from the group consisting of SEQ ID NOs: 2, 4, 5, 7, 9, 11 and 13 or a complement of said nucleic acid sequence in the tissue or at least one mast cell to the level of expression of the nucleic acid sequence in the database.

66. A method of claim 64 or 65, wherein the expression level of at least two nucleic acid sequences are compared.

67. A method of claim 66, wherein the expression level of at least five nucleic acid sequences are compared.

68. A method of claim 67, wherein the expression level of all the nucleic acid sequences are compared.

69. A computer system comprising:
the nucleotide and/or amino acid sequence of at least one of SEQ ID NOs: 1-13 and a user interface to view the information.
